(19) United States
(12) Patent Application Publication
(10) Pub. No.: US 2023/0301673 A1
(43) Pub. Date: Sep. 28, 2023

(54) SYSTEMS AND METHODS FOR TREATMENT OF DEFECTS IN THE VASCULATURE WITH A STENT RETRIEVER

(71) Applicant: Neuravi Limited, Galway (IE)

(72) Inventors: Emilie KOTTENMEIER PARK, Los Angeles, CA (US); Patrick BROUWER, Haarlem (NL); Shelly IKEME, West Chester, PA (US); Alia KHALED, Burlington (CA); Mina KABIRI, Auston, TX (US); Mahmood MIRZA, Galway (IE); Eamon BRADY, Loughrea (IE); David VALE, Barna (IE); Michael GILVARRY, Headford (IE); Mahmood K. RAZAVI, Irvine, CA (US); John O’SHAUGHNESSY, Galway (IE); Brendan CASEY, Galway (IE); David HARDIMAN, Dublin (IE); Kevin McARDLE, Loughrea (IE); Patrick GRIFFIN, Castlegar (IE); Jason McNAMARA, Knock (IE)

(73) Assignee: Neuravi Limited, Galway (IE)

(21) Appl. No.: 18/104,493

(22) Filed: Feb. 1, 2023

Related U.S. Application Data
(60) Provisional application No. 63/398,378, filed on Aug. 16, 2022.
(60) Provisional application No. 63/307,697, filed on Feb. 8, 2022.

(51) Int. Cl. A61B 17/221

(52) U.S. Cl. CPC A61B 17/221; A61B 2017/2212

(57) ABSTRACT

Method for using a clot retrieval device for treating a clot in a blood vessel for use in the treatment of ischemic stroke to reperfuse an obstructed vessel. The method comprising delivering a revascularization device to a blood vessel of a respective human patient of a first plurality of human patients for retrieving a clot and removing the revascularization device and restoring perfusion to the blood vessel for the first plurality of human patients with one or more clots by passing the revascularization device by, through, or about the clot to achieve a reperfusion outcome greater than approximately 53.5% under a modified Rankin Scale (mRS) score of 0-2 within a predetermined time period.

### CROSS-REFERENCE TO RELATED APPLICATIONS

This application claims the benefit of U.S. Provisional Pat. Application No. 63/307,697, filed Feb. 8, 2022, which is incorporated by reference herein in its entirety. This application also claims the benefit of U.S. Provisional Pat. Application No. 63/398,378, filed Aug. 16, 2022, which is incorporated by reference herein in its entirety.

### FIELD

This disclosure relates to devices and methods of removing acute blockages from blood vessels.

### BACKGROUND

The World Health Organization estimates that 15,000,000 blood clots occur annually. Clots may develop and block vessels locally without being released in the form of an embolus—this mechanism is common in the formation of coronary blockages. Acute obstructions may include blood clots, misplaced devices, migrated devices, large emboli and the like. Thromboembolism occurs when part or all of a thrombus breaks away from the blood vessel wall. This clot is then carried in the direction of blood flow. The large vessels of the brain include the Internal Carotid Artery (ICA), Middle Cerebral Artery (MCA), Vertebral Artery (VA), and the Basilar Artery (BA). Clots can include a range of morphologies and consistencies. Long strands of softer clot material may tend to lodge at bifurcations or trifurcations, resulting in multiple vessels being simultaneously occluded over significant lengths. Older clot material can also be less compressible than softer fresher clots, and under the action of blood pressure it may distend the compliant vessel in which it is lodged. Clots may also vary greatly in length, even in any one given area of the anatomy. For example, clots occluding the middle cerebral artery of an ischemic stroke patient may range from just a few millimeters to several centimeters in length.

Of the 15,000,000 clots that occur annually, one-third of patients die and another one-third are disabled. Two of the primary factors associated with mortality in these patients are the occlusion location and the time to treatment. Large-vessel occlusions present in 46% of unselected acute stroke patients presenting in academic medical centers, are associated with higher stroke severity. These more proximal vessels feed a large volume of brain tissue, ergo clinicians use the presenting NIHSS (National Institute of Health Stroke Scale) score as an indicator of large-vessel occlusion.

With this, it is understood that an ischemic stroke may result if the clot lodges in the cerebral vasculature. It is estimated that 87% of stroke cases are acute ischemic stroke (AIS). In the United States alone, roughly 700,000 AIS cases occur every year and this number is expected to increase with an ageing population. Occlusion of these large arteries in ischemic stroke is associated with significant disability and mortality. Revascularization of intracranial artery occlusions is the therapeutic goal in stroke therapy. Endovascular mechanical revascularization (thrombectomy) is an increasingly used method for intracranial large vessel recanalization in acute stroke. Currently, a number of mechanical recanalization devices are in clinical use. First generation devices included the Merci Retriever device. Newer devices based on stent-like technology, referred to as “stentrievers” or “stent-retrievers”, are currently displacing these first generation thrombectomy devices for recanalization in acute ischemic stroke.

Several randomized clinical trials have demonstrated that mechanical thrombectomy using stent-like clot retriever devices are a safe and effective treatment to remove clots from cerebral vessels of acute stroke patients, but such devices are not without disadvantages. A stent-like clot retriever relies on its outward radial force to grip the clot. If the radial force is too low, the device will lose its grip on the clot. If the radial force is too high, the device may damage the vessel wall and may require too much force to withdraw. Such devices that have sufficient radial force to deal with all clot types may therefore cause vessel trauma and serious patient injury, and retrievers that have appropriate radial force to remain atraumatic may not be able to effectively handle all clot types. In this respect, retriever devices may differ in size, shape, and physical properties, such as radial force, as discussed above, ease of deployment, friction, radiopacity and interaction with vessel wall. See, Loh Y, Jahan R, McArthur D. Recanalization rates decrease with increasing thrombectomy attempts. American Journal of Neuroradiology. 2010 May;31(5):935-9; and Arai D, Ishii A, Chihara H, Ikeda H, Miyamoto S. Histological examination of vascular damage caused by stent retriever thrombectomy devices, J Neurointerv Surg. 2016 Oct;8(10):992-5. Some designs have also been based on in-vitro stroke models that incorporate realistic clot analogs derived from animal blood that represent the wide range of human clots retrieved from stroke patients. See, Eugène F, Gauvrit J-Y, Ferré J-C, Gentric J-C, Besseghir A, Ronzière T, et al. One-year MR angiographic and clinical follow-up after intracranial mechanical thrombectomy using a stent retriever device, AJNR American Journal of Neuroradiology. 2015 Jan;36(1):126-32 (18), each of which are incorporated by reference herein in their entirety.

Currently, intravenous (IV) lytics are used for patients presenting up to 4.5 hours after symptom onset. Current guidelines recommend administering IV lytics in the 3-4.5 hour window to those patients who meet the ECASS 3 (European Cooperative Acute Stroke Study 3) trial inclusion/exclusion criteria. Since a large percentage of strokes presenting at hospitals are large vessel occlusions, this is an important clinical challenge to address. Additionally, not all patients may be treated with thrombolytic therapy, and so mechanical thrombectomy is a valuable alternative in patients contraindicated to tPA (tissue plasminogen activator) or where tPA treatment was not effective.

Further, acute stroke treatment protocols vary by hospital center. Often, CT is used to exclude hemorrhagic stroke, and CT Angiography is used. Additional imaging assessment, such as MRI or CT Perfusion, varies by center. Recent AIS trials have demonstrated the clinical benefit and reperfusion efficacy of endovascular therapy using stent-retriever devices. See Zaidat OO, Castonguay AC, Gupta R, Sun CJ, Martin C, Holloway WE, et al. The first pass effect: a new measure for stroke thrombectomy devices. J NeuroIntervent Surg. 2015;7(suppl 1):A2-A3; Chueh JY, Marosfoi MG, Brooks OW, King RM, Puri AS, Gounis MJ. Novel distal emboli protection technology: the EmboTrap. Interv Neurol. 2017;6:268-276. doi: 10.1159/000480668; Kabbasch C, Mpotsaris A, Liebig T, Söderman M, Holtmannspötter M, Cronqvist M, et al. TREVO 2 Trialists. Trevo versus Merci retrievers for thrombectomy revascularisation of large vessel occlusions in acute ischaemic stroke (TREVO 2): a randomised trial. Lancet. 2012;380:1231-1240. doi: 10.1016/S0140-6736(12)61299-9. There are several FDA approved stent retriever devices indicated for neuro-thrombectomy, including Merci®, Trevo®, and Solitaire®. These devices are generally described in U.S. Pat. Nos. 8,066,757; 8,088,140; 8,945,172; 9,320,532; 8,585,713; 8,945,143; 8,197,493; 8,940,003; 9,161,766; 8,679,142; 8,070,791; 8,574,262; 9,387,098; 9,072,537; 9,044,263; 8,795,317; 8,795,345; 8,529,596; and 8,357,179. Presently, these devices are now considered the standard of care for treatment of AIS secondary to large-vessel occlusion. See, Powers WJ, Derdeyn CP, Biller J, Coffey CS, Hoh BL, Jauch EC, et al; American Heart Association Stroke Council. 2015 American Heart Association/American Stroke Association focused update of the 2013 guidelines for the early management of patients with acute ischemic stroke regarding endovascular treatment: a guideline for healthcare professionals from the American Heart Association/American Stroke Association. Stroke. 2015; 46:3020-3035. doi: 10.1161/STR.0000000000000074.

Two multi-center studies using a specific FDA approved thrombectomy device generally described in U.S. Pat. Nos. U.S. Pat. Nos. 8,777,976; 8,852,205; 9,402,707; 9,445,829; and 9,642,639, demonstrated high reperfusion rates (modified treatment in cerebral ischemia (mTICI) ≥ 2b within three passes without rescue) of 75.0% and 80.2%, respectively. See Zaidat OO, Bozorgchami H, Ribo M, et al. Primary Results of the Multicenter ARISE II Study (Analysis of Revascularization in Ischemic Stroke With EmboTrap). Stroke 2018;49:1107-15; Mattle HP, Scarrott C, Claffey M, et al. Analysis of revascularisation in ischaemic stroke with EmboTrap (ARISE I study) and meta-analysis of thrombectomy. Interv Neuroradiol. 2019;25:261-70. These trials had prespecified patient inclusion and exclusion criteria, and thus patient outcomes may differ from real-world use of the stent retriever device used in those studies. Two U.S. studies assessing hospital outcomes among patients who underwent endovascular treatment for acute ischemic stroke using two other thrombectomy devices using real-world data reported outcomes parameters such as hospital length of stay (LOS) and hospital costs. See Rai AT, Crivera C, Kalsekar I, et al. Endovascular Stroke Therapy Trends From 2011 to 2017 Show Significant Improvement in Clinical and Economic Outcomes. Stroke. 2019a;50:1902-06; Rai AT, Crivera C, Kottenmeier E, et al. Outcomes associated with endovascular treatment among patients with acute ischemic stroke in the USA. J Neurointerv Surg. 2019b;12:422-26.

No studies have assessed relative advantages of different FDA approved thrombectomy devices in patients with AIS to compare respective mortality, recanalization, functional, and safety outcomes associated with the use of such devices. The solution of this disclosure resolves these and other issues of the art.

### SUMMARY

The subject of this disclosure is the use of a clot retrieval device to treat ischemic stroke for restoring perfusion and/or removing a clot and other obstructions from the neurovascular arteries and veins as well as other vascular beds.

An example method of restoring blood flow in neurovasculature can include removing a clot in human patients experiencing ischemic stroke by delivering a revascularization device to a blood vessel of a respective human patient of a first plurality of human patients for retrieving a clot and removing the revascularization device and restoring perfusion to the blood vessel by passing the revascularization device by, through, or about the clot of the blood vessel. The method can achieve, by the revascularization device, a reperfusion outcome greater than approximately 53.5% under a modified Rankin Scale (mRS) score of 0-2 within a predetermined time period.

An example method can further include achieving, by the revascularization device, a reperfusion outcome less than or equal to approximately 64.4% under a modified Rankin Scale (mRS) score of 0-2 within a predetermined time period.

An example predetermined time period can be within 90-days following restoring perfusion to the blood vessel.

An example revascularization device can include an inner tubular body having a plurality of openings, a collapsed delivery configuration, and an expanded deployed configuration. The revascularization device can also include an outer tubular body at least partially overlying the inner tubular body and having a plurality of closed cell.

An example method can further include achieving a reperfusion outcome greater than approximately 19.1% under a mRS score of 0 for the first plurality of human patients at 90-days following restoring perfusion to the blood vessel.

An example method can further include achieving a reperfusion outcome of at least approximately 21.1% under a mRS score of 0 for the first plurality of human patients at 90-days following restoring perfusion to the blood vessel.

An example method can further include achieving a reperfusion outcome greater than approximately 15.5% under a mRS score of 2 for the first plurality of human patients at 90-days following restoring perfusion to the blood vessel.

An example method can further include achieving a reperfusion outcome of at least approximately 20.6% under a mRS score of 2 for the first plurality of human patients at 90-days following restoring perfusion to the blood vessel.

An example method can further include delivering a first comparative revascularization device to a blood vessel of a respective human patient of a second plurality of human patients with one or more clots and restoring perfusion to the respective blood vessel by passing the first comparative revascularization device by, through, or about the respective clot of the blood vessel. The method can further include achieving, by the revascularization device, a decreased average symptomatic intracranial hemorrhage (sICH) outcome for the first plurality of human patients by approximately 0.7% compared to the second plurality of human patients.

An example method can further include achieving, by the revascularization device, an increased average reperfusion outcome under a mRS score of 0-2 at 90-days following restoring perfusion to the blood vessel for the first plurality of human patients by approximately 7.4%, by the revascularization device compared to the second plurality of human patients.

An example method can further include achieving, by the revascularization device, a reduced average mortality at 90-days following restoring perfusion to the blood vessel for the first plurality of human patients by approximately 3.3% compared to the second plurality of human patients.

An example method can further include delivering a second comparative revascularization device to a blood vessel of a respective human patient of a third plurality of human patients with one or more clots and restoring perfusion to the respective blood vessel by passing the second comparative revascularization device by, through, or about the respective clot of the blood vessel. The method can further include achieving, by the revascularization device, a decreased average symptomatic intracranial hemorrhage (sICH) outcome for the first plurality of human patients by approximately 3.8% compared to the third plurality of human patients.

An example method can further include achieving, by the revascularization device, an increased average reperfusion outcome under a mRS score of 0-2 at 90-days following restoring perfusion to the blood vessel for the first plurality of human patients by approximately 12.1%, by the revascularization device compared to the third plurality of human patients.

An example method can further include achieving, by the revascularization device, a reduced average mortality at 90-days following restoring perfusion to the blood vessel for the first plurality of human patients by approximately 9.2% compared to the third plurality of human patients.

An example method can include a population size for the first plurality of human patients having at least 633 patients.

An example revascularization device for restoring perfusion to a blood vessel of a respective human patient of a first population of human patients can include a device having a collapsed delivery configuration and an expanded deployed configuration. The device can further include a framework of struts forming a porous outer body radially surrounding a porous inner body during both the collapsed delivery configuration and the expanded deployed configuration, the outer body being expandable to a radial extent to define a clot reception space. The device can also include a distal end of the inner body located within the outer body and adjacent a distal end of the outer body and extending in the deployed configuration towards the outer body to a greater extent than the inner body. The revascularization device can be capable of being delivered to a respective blood vessel of the first population of human patients and restoring perfusion to the blood vessel by passing the revascularization device by, though, or about a clot to achieve a reperfusion outcome greater than approximately 53.5% and less than or equal to 64.4% under a modified Rankin Scale (mRS) score of 0-2 at 90-days following restoring perfusion to the blood vessel.

An example revascularization device can further be capable of being delivered to a blood vessel and restoring perfusion to the blood vessel by passing the revascularization device by, though, or about the clot to achieve a mean reperfusion outcome under a modified Rankin Scale (mRS) score of at least 1.63 for the first population of human patients at 90-days following restoring perfusion to the blood vessel. The mean reperfusion outcome can be for a first population of at least 475 human patients.

An example revascularization device can further be capable of being delivered to a blood vessel and restoring perfusion to the blood vessel by passing the revascularization device by, though, or about the respective clots to achieve a decreased mean reperfusion outcome under a modified Rankin Scale (mRS) score at 90-days following restoring perfusion to the respective blood vessel within a first population of human patients by approximately 0.82 compared to a mean reperfusion outcome under the mRS score at 90-days following reperfusion, by passing a first comparative revascularization device within a second population of human patients. The mean reperfusion outcome for the first population of human patients can include at least 475 patients and wherein the mean reperfusion outcome for the second of human patients can include at least 2598 patients.

An example revascularization device can further be capable of being delivered to a blood vessel and restoring perfusion to the blood vessel by passing the revascularization device by, though, or about the respective clot to achieve a decreased mean reperfusion outcome under a modified Rankin Scale (mRS) score at 90-days following restoring perfusion to the respective blood vessel within a first population of human patients by approximately 1.1 compared to a mean reperfusion outcome under the mRS score at 90-days following reperfusion, by passing a second comparative revascularization device within a third population of human patients. The mean reperfusion outcome for the first population of human patients can include at least 475 patients and wherein the mean reperfusion outcome for the third population of human patients can include at least 2893 patients.

An example method of restoring blood flow in neurovasculature can include removing a clot in human patients experiencing ischemic stroke by delivering a revascularization device to a blood vessel of a respective human patient of a first population of human patients for retrieving a clot and removing the revascularization device. The method can include restoring perfusion to the blood vessel for the first population of human patients with one or more clots by passing the revascularization device by, through, or about the clot. The method can further include achieving a mortality outcome at 90-days following restoring perfusion to the blood vessel for the first population of human patient of less than or equal to approximately 11.4%.

An example method can further include achieving a mean symptomatic intracranial hemorrhage (sICH) outcome of approximately 3.9% for the first population of human patients.

An example revascularization device can include an inner tubular body having a plurality of openings, a collapsed delivery configuration, and an expanded deployed configuration. The revascularization device can also include an outer tubular body at least partially overlying the inner tubular body and having a plurality of closed cell.

An example method can further include achieving a mean reperfusion outcome under a modified Rankin Scale (mRS) score of at least 1.63 for the first population of human patients at 90-days following restoring perfusion to the blood vessel.

An example method can further include achieving a decreased mean reperfusion outcome under a modified Rankin Scale (mRS) score at 90-days following restoring perfusion to the respective blood vessel within the first population of human patients by approximately 0.82 compared to a mean reperfusion outcome under the mRS score at 90-days following reperfusion, by passing a first comparative revascularization device within a second population of human patients.

An example method can further include achieving a decreased mean reperfusion outcome under a modified Rankin Scale (mRS) score at 90-days following restoring perfusion to the respective blood vessel within the first population of human patients by approximately 1.1 compared to a mean reperfusion outcome under the mRS score at 90-days following reperfusion, by passing a second comparative revascularization device within a third population of human patients.

An example method can further include achieving a decreased mean reperfusion outcome under a modified Rankin Scale (mRS) score at 90-days following restoring perfusion to the respective blood vessel within the first population of human patients by approximately 1.1 compared to a mean reperfusion outcome under the mRS score at 90-days following reperfusion, by passing a second comparative revascularization device within a third population of human patients.

To the accomplishment of the foregoing and related ends, certain illustrative aspects are described herein in connection with the following description and the appended drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 shows a patient catheterized via femoral access with an example clot retrieval device positioned in a cerebral vessel using the arterial system for its delivery.

FIG. 2 shows certain anatomy of cerebral arteries above the aortic arch leading to the brain.

FIG. 3 shows an isometric view of an example stent retriever device of this disclosure.

FIG. 4 is a table summarizing Thrombolysis in Cerebrovascular Infarction (mTICI) inclusive of the 2c rating for the study of this disclosure.

FIG. 5 is a table summarizing Modified Rankin Scale (mRS) score for the study of this disclosure.

FIG. 6 is a flow diagram illustrating flow of enrollment of records for data extraction for the study of this disclosure.

FIGS. 7 and 8 are tables summarizing background characteristics of patients treated with one of the revascularization devices of the study of this disclosure.

FIGS. 9 and 10 are tables of functional and safety outcomes between patients treated with one of the revascularization devices of the study of this disclosure.

FIG. 11 is a table summarizing the statistical comparison of functional and safety outcomes between patients treated with one of the revascularization devices of the study of this disclosure.

FIG. 12 is a table of functional and safety outcomes based on a core lab adjudication between patients treated with one of the revascularization devices of the study of this disclosure.

FIG. 13 is a table summarizing the statistical comparison of functional and safety outcomes based on a core lab adjudication between patients treated with one of the revascularization devices of the study of this disclosure.

FIG. 14 depicts a flowchart overview of a method of treating thrombus by mechanical thrombectomy according to this disclosure.

FIG. 15 depicts a flowchart overview of a method of treating thrombus by mechanical thrombectomy according to this disclosure.

FIG. 16 depicts a flowchart overview of a method of treating thrombus by mechanical thrombectomy according to this disclosure.

FIG. 17 depicts a flowchart overview of a method of treating thrombus by mechanical thrombectomy according to this disclosure.

### DETAILED DESCRIPTION

Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 99%.

As discussed herein, vasculature of a “subject” or “patient” may be vasculature of a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey, or the like). It should be appreciated that the subject may be any applicable human patient, for example.

As discussed herein, “operator” may include a doctor, surgeon, or any other individual or delivery instrumentation associated with delivery of a clot retrieval device to the vasculature of a subject.

As discussed herein, “thrombus” can be understood as a clot in the circulatory system that remains in a site of the vasculature hindering or otherwise obstructing flow in a blood vessel. The terms, “clot”, “thrombus”, “obstruction”, “occlusion”, “blockage”, and/or the like, can be and are often used interchangeably throughout this disclosure.

Delivery of a “revascularization device” is typically accomplished via delivery of one or more catheters into the femoral artery and/or the radial artery, guided into the arteries of the brain, vascular bypass, angioplasty, and/or the like. “Revascularization devices” can include, but not be limited to, one or more stents, stentrievers, clot removal devices, clot retrieval devices, aspiration systems, one or more combinations thereof, and/or the like, each of which are often used interchangeably throughout this disclosure.

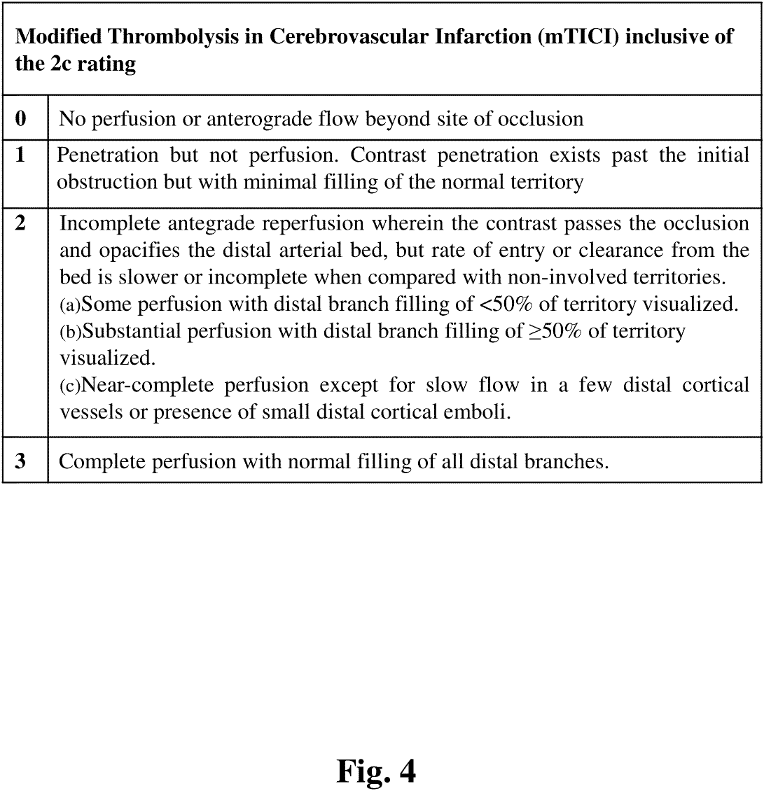

As discussed herein and provided in FIG. 4, “mTICI” means modified thrombolysis in cerebral infarction (TICI) score. An mTICI score of 0 means no perfusion. An mTICI score of 1 means antegrade reperfusion past the initial occlusion but limited distal branch filling with little or slow distal reperfusion. An mTICI score of 2 generally means incomplete antegrade reperfusion wherein the contrast passes the occlusion and opacifies the distal arterial bed but there are residual antegrade perfusion deficits. More particularly, an mTICI score of 2a means antegrade reperfusion of less than half of the occluded target artery previously ischemic territory (e.g., in 1 major division of the MCA and its territory). An mTICI score of 2b means antegrade reperfusion of more than half of the previously occluded target artery ischemic territory (e.g., in 2 major divisions of the MCA and their territories). An mTICI score of 2c means antegrade reperfusion of >90% but less than TICI 3 or near complete reperfusion. An mTICI score of 3 means full perfusion with filling of all distal branches.

It is noted, however, that other measures of cerebral scoring standards, such as expanded TICI (eTICI), other known and/or to-be-developed cerebral scoring standards, provide measures of cerebral scoring and are thus directly and/or indirectly applicable in understanding scope of the presently disclosed solution. eTICI scale is a 7-point compilation of TICI grades that reflects all previously reported thresholds used to define reperfusion after endovascular stroke therapy. For example, eTICI grade 0, just as mTICI, can be equivalent to no reperfusion or 0% filling of the downstream territory. eTICI 1 can indicate thrombus reduction without any reperfusion of distal arteries, including reperfusion of less than half or 1-49%. eTICI of 2b50 can be 50-66% reperfusion. eTICI 2b67 can be 67-89% reperfusion, exceeding TICI but below TICI2C. eTICI 2c can be equivalent to TICI 2C or 90-99% reperfusion. eTICI 3 can be complete or 100% reperfusion, such as TICI 3. It is understood that one of ordinary skill in the art can also correlate between currently known cerebral scoring standards and/or to-be-developed cerebral scoring standards (e.g., from mTICI to eTICI).

As discussed herein, “NIHSS Score” means The National Institutes of Health Stroke Scale, or NIH Stroke Scale (NIHSS) and is a tool used by healthcare providers to objectively quantify the impairment caused by a stroke. The NIHSS is composed of 11 items, each of which scores a specific ability between a 0 and 4. For each item, a score of 0 typically indicates normal function in that specific ability, while a higher score is indicative of some level of impairment. The individual scores from each item are summed in order to calculate a patient’s total NIHSS score. The maximum possible score is 42, with the minimum score being a 0.

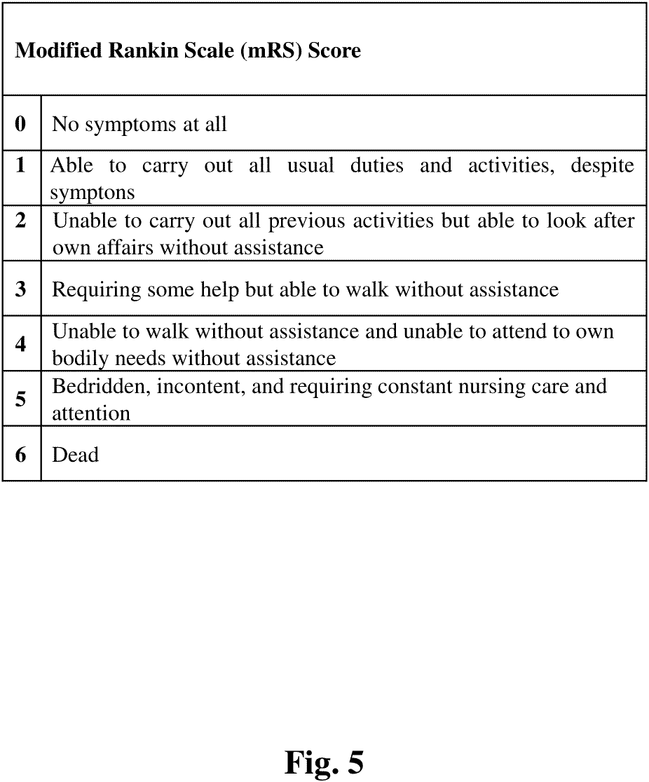

As discussed herein and provided in FIG. 5, “mRS” means the modified Rankin Scale (mRS) that is a commonly used scale for measuring the degree of disability or dependence in the daily activities of people who have suffered a stroke or other causes of neurological disability. The mRS scale runs from 0-6, running from perfect health without symptoms (0) to death (6). An mRS score of 0 is understood as no symptoms being observed. An mRS score of 1 is understood as no significant disability is observed and the patient is able to carry out all usual activities, despite some symptoms. An mRS score of 2 is understood as slight disability and the patient is able to look after own affairs without assistance, but unable to carry out all previous activities. An mRS score of 3 is understood as moderate disability whereby the patient can require some help but is able to walk unassisted. An mRS score of 4 is understood as moderate severe disability and the patient is unable to attend to own bodily needs without assistance or walk unassisted. An mRS score of 5 is understood as severe disability and the patient requires constant nursing care and attention, bedridden, incontinent. An mRS score of 6 is understood as the patient being deceased.

As discussed herein, the term “safety”, as it relates to a clot retrieval device, delivery system, or method of treatment refers to a relatively low severity of adverse events, including adverse bleeding events, infusion or hypersensitivity reactions. Adverse bleeding events can be the primary safety endpoint and include, for example, major bleeding, minor bleeding, and the individual components of the composite endpoint of any bleeding event.

As discussed herein, unless otherwise noted, the term “clinically effective” (used independently or to modify the term “effective”) can mean that it has been proven by a clinical trial wherein the clinical trial has met the approval standards of U.S. Food and Drug Administration, EMEA or a corresponding national regulatory agency. For example, a clinical study may be an adequately sized, randomized, double-blinded controlled study used to clinically prove the effects of the reperfusion device and related systems of this disclosure. Most preferably to clinically prove the effects of the reperfusion device with respect to an ischemic event, for example, to achieve a clinically effective outcome in for the patient suffering the ischemic event (e.g., mRS less than or equal to 2) and/or achieve reperfusion the vessel(s) afflicted by the ischemic event.

As discussed herein, symptomatic intracranial hemorrhage, or “sICH” is any extravascular blood in the brain or within the cranium associated with clinical deterioration, as defined by an increase of 4 points or more in the score on the NIHSS, or that leads to death and is identified as the predominant cause of the neurologic deterioration. For the purpose of this disclosure, subjects with sICH identified through all post-treatment scans up to the 24-hour time-point (including those performed due to clinical deterioration), were considered in the study discussed herein.

As discussed herein, the term “computed tomography” or CT means one or more scans that make use of computer-processed combinations of many X-ray measurements taken from different angles to produce cross-sectional (tomographic) images (virtual “slices”) of specific areas of a scanned object, allowing the user to see inside the object without cutting. Such CT scans of this disclosure can refer to X-ray CT as well as many other types of CT, such as positron emission tomography (PET) and single-photon emission computed tomography (SPECT).

As used herein, the term “odds ratio” or “OR” means the strength of an association between two events. In general, two events are independent if and only if the OR equals 1.0 (e.g., the ratio of odds of one event are the same in either the presence or absence of the other event).

The present disclosure is related to systems, methods and devices restoring perfusion in blood vessels, and in particular clots from cerebral vessels. Certain features, such as a capture net, can be designed to trap a wide range of clot compositions inside the device, and an inner channel to stabilize the clot during retrieval. Certain feature of the retriever of this disclosure can allow the segments to remain open and opposed to the vessel wall while retracted through challenging vessels.

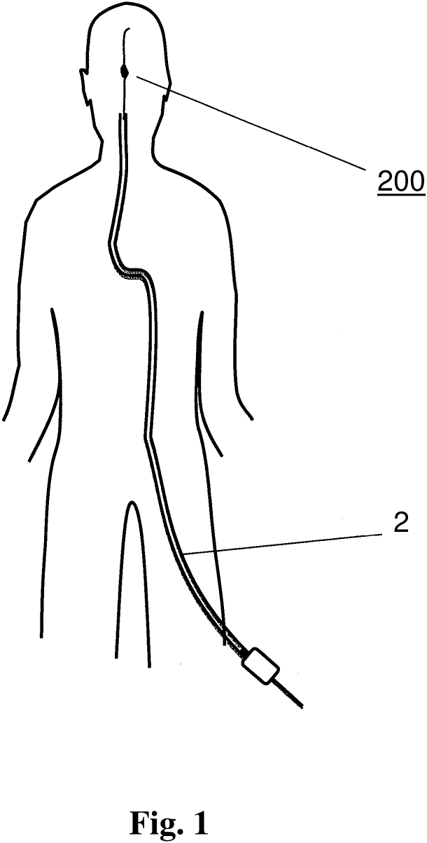

As an example, FIG. 1 depicts a schematic representation of the catheterization of a patient with a clot retrieval device 200, also known as a reperfusion device, via the femoral artery with a catheter 2. Example device 200 is a clinically approved FDA clot retrieval device that can restore blood flow in the neurovasculature by removing thrombus in patients experiencing ischemic stroke within 8 hours of symptom onset. However, it is understood that example device 200 could be used to restore blood flow in less than 8 hours of symptom onset (e.g., 6 hours) or up to 24 hours from symptom onset. As applicable procedure guidelines change with respect to the use of clot retrieval devices for treatment of ischemic events, it is also conceivable that device 200 could be used more than 24 hours from symptom onset. Device 200 can be understood as including features are clearly described in Appendix 1, which is included in U.S. Provisional Application No. 63/307,697, filed Feb. 8, 2022 and in U.S. Provisional Pat. Application No. 63/398,378, filed Aug. 16, 2022 from which this Non-Provisional claims priority in Paragraph [0001] of this application, and as incorporated by reference in its entirety from U.S. Pat. Nos. 8,777,976; 8,852,205; 9,402,707; 9,445,829; and 9,642,639, each of which are incorporated by reference in their entirety as if set forth verbatim herein. Note that reperfusion devices can also be introduced through the wrist artery (radial access) or directly through the carotid artery. While both radial and carotid access avoids the aortic arches, there are other drawbacks. However, all three approaches are considered to be known to ones of skill in this art.

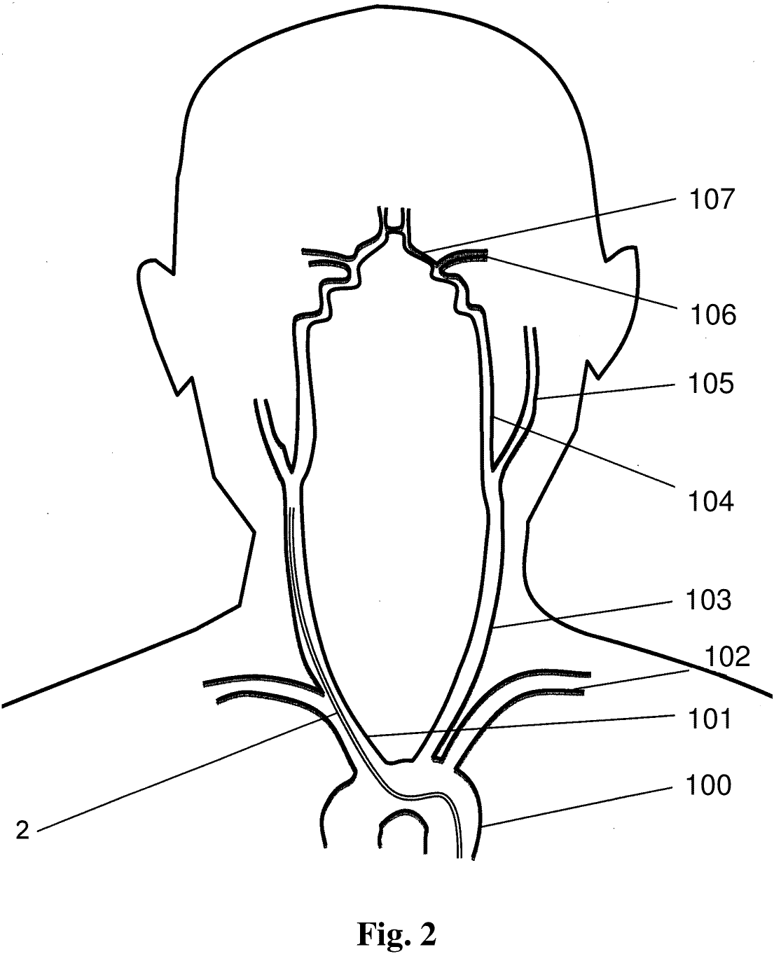

FIG. 2 shows a schematic representation of certain example cerebral vessels. Vessel 100 is the Aorta. Vessel 101 is the brachiocephalic artery. Vessel 102 is the subclavian artery. Vessel 103 is the common carotid artery. Vessel 104 is the internal carotid artery. Vessel 105 is the external carotid artery. Vessel 106 is the middle cerebral artery. Vessel 107 is the anterio-cerebral artery. The catheter 2 from FIG. 1 is shown with its distal end in the common carotid artery. In the more detailed drawings of the invention the details of the access site will not be shown but in general access and delivery is in accordance with FIGS. 1 and 2. Device 200 can be designed for use in the anterior and posterior neurovasculature in vessels such as the internal carotid artery, the M1 and M2 segments of the middle cerebral artery, the vertebral artery, and the basilar arteries. Device 200 can be delivered endovascularly under fluoroscopic guidance in a similar manner to that of other neurovascular clot-retrieval systems.

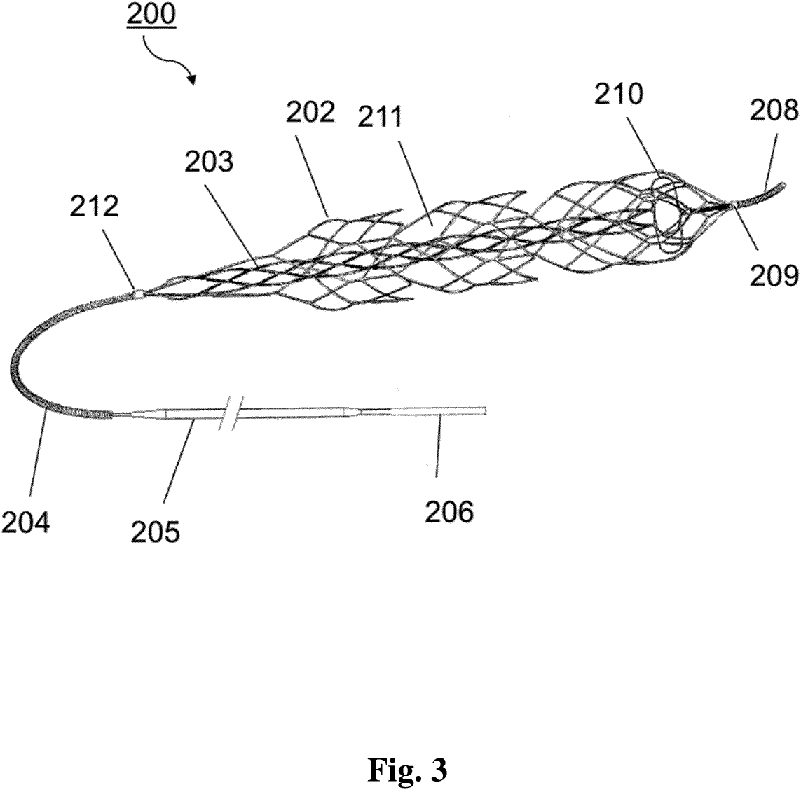

Once across the site of vessel occlusion, the stent-like element of device 200 is deployed to entrap the clot and allow it to be retrieved, hence restoring blood flow. Device 200 can be a dual-layer stent retriever, with articulating petals, and a distal capture zone for effectively trapping, retaining, and removing various clot types to restore blood flow in patients with AIS secondary to large-vessel occlusion. Examples of the device 200 can be available in two lengths, 5x21 mm and 5x33 mm. It is understood that device 200 of this disclosure would be used with a delivery system to the site of the clot, including a guide catheter, a microcatheter, and/or a guidewire. It is also contemplated that device 200 of this disclosure could be used in connection with an aspiration system to further facilitate restoring perfusion to the vasculature. FIG. 3 shows one embodiment of an example clot retrieval device of this disclosure. Device 200 can have an elongate shaft 206. Shaft 206 can have a distal end that extends interior of the artery and a proximal end that extends exterior of the artery. Shaft 206 can also have a clot engaging portion configured at its distal end having an outer expandable member 202 and an inner expandable member 203 to facilitate restoration of blood flow through the clot after device 200 is deployed. Members 202 and 203 can be configured to have a collapsed configuration for delivery and an expanded configuration for clot retrieval, restoration of perfusion, and fragmentation protection in general.

Shaft 206 may be a tapered wire shaft, and may be made of stainless steel, MP35N, Nitinol or other material of a suitably high modulus and tensile strength. Shaft 206 has a coil 204 adjacent its distal end and proximal of the outer member and inner tubular member. The coil may be coated with a low friction material or have a polymeric jacket positioned on the outer surface. Sleeve 205 may be positioned on shaft 206 adjacent coil 204. Sleeve 205 may be polymeric and may be positioned over the tapered section of shaft 206.

The outer member 202 is configured to self-expand upon release from a microcatheter to a diameter larger than that of the inner tubular member 203. Expansion of the outer member 202 causes compression and/or displacement of the clot during expansion for purposes of restoring perfusion to the vessel. A radiopaque coil 208 (which may be platinum or gold or an alloy of same) is positioned over the distal end of member 203 and butts against the distal collar 209 of the outer member 202, where it is connected by an adhesive joint to the collar 209. In some examples, the distal end of device 200 at or adjacent collar 209 can be closed by way of struts 210 being joined. In some examples, the outer member 202 can have a closed distal clot capture structure whereby a plurality of struts converge at a terminal connection. In some examples, the distal end of the outer member 202 can have its struts terminate at a distal end in a junction to define a closed end that can prevent egress of clot (or clot fragments that have entered thereof) between the inner 203 and outer 202 members. Inlet openings of outer member 202 can provide the primary movement freedom available to the clot and so the expansion of the outer member 202 urges the clot into the reception space 211 and outer member 202 can have multiple inlet mouths to accept the clot. Optionally expanded distal struts 210 can be included with the inner member 203 and function as an additional three-dimensional filter to prevent the egress of clot or clot fragments.

### Study Overview

This disclosure is more clearly understood with the following fifty-one corresponding studies discussed more particularly below with respect to treatment of acute ischemic stroke:
1. Baek J-H, Joonsang Y, Dongbeom S, et al. Predictive value of thrombus volume for recanalization in stent retriever thrombectomy. Scientific Reports 2017;7(None) doi: 10.1038/s41598-017-16274-9;
2. Baek, Jin Wook, Young Jin Heo, Sung Tae Kim, Jung Hwa Seo, Hae Woong Jeong, and Eung-Gyu Kim. Comparison of the Solitaire and Trevo Stents for Endovascular Treatment of Acute Ischemic Stroke: A Single. Center Experience. Neurol India 69, no. 2 (Jan. 1, 2021);
3. Binning, Mandy J, Bruno Bartolini, Blaise Baxter, Ronald Budzik, Joey English, Rishi Gupta, Hirad Hedayat, et al. Trevo 2000: Results of a Large Real-World Registry for Stent Retriever for Acute Ischemic Stroke. J Am Heart Assoc 7, no. 24 (Dec. 18, 2018);
4. Bourcier R, Abed D, Piotin M, et al. Multicenter initial experience with the EmboTrap device in acute anterior ischemic stroke. Journal of neuroradiology 2018;45(4) doi: 10.1016/j.neurad.2018.01.052
5. Brouwer, Patrick A, Leonard L Yeo, Ake Holmberg, Tommy Andersson, Jens Kolloch, Åsa KuntzeSöderqvist, Marcus Ohlsson, et al. Thrombectomy Using the EmboTrap Device: Core Laboratory-Assessed Results in 201 Consecutive Patients in a Real-World Setting. Journal of Neurointerventional Surgery 10, no. 10 (Oct. 1, 2018);
6. Cabral, Norberto L, Adriana Conforto, Pedro S C Magalhaes, Alexandre L Longo, Carla H C Moro, Hamilton Appel, Paulo Wille, et al. Intravenous RtPA versus Mechanical Thrombectomy in Acute Ischemic Stroke: A Historical Cohort in Joinville, Brazil. ENeurologicalSci 5, no. None (Dec. 1, 2016);
7. Campbell, Bruce C V, Peter J Mitchell, Timothy J Kleinig, Helen M Dewey, Leonid Churilov, Nawaf Yassi, Bernard Yan, et al. Endovascular Therapy for Ischemic Stroke with Perfusion-Imaging Selection. N Engl J Med 372, no. 11 (Mar. 12, 2015);
8. Cao, Jie, Hang Lin, Min Lin, Kaifu Ke, Yunfeng Zhang, Yong Zhang, Weihong Zheng, et al. RECO Flow Restoration Device Versus Solitaire FR With the Intention for Thrombectomy Study (REDIRECT): A Prospective Randomized Controlled Trial. J Neurosurg None, no. None (Jun. 5, 2020);
9. Choi, Jae-Hyung, Hyun-Seok Park, Dae-Hyun Kim, Jae-Kwan Cha, Jae-Taeck Huh, and Myongjin Kang. Comparative Analysis of Endovascular Stroke Therapy Using Urokinase, Penumbra System and Retrievable (Solitare) Stent. J Korean Neurosurg Soc 57, no. 5 (May 1, 2015);
10. Guo, Yongtao, Wenjie Zi, Yue Wan, Shuai Zhang, Bo Sun, Xianjin Shang, Shun Li, et al. Leukoaraiosis Severity and Outcomes after Mechanical Thrombectomy with Stent-Retriever Devices in Acute Ischemic Stroke. J Neurointerv Surg 11, no. 2 (Feb. 1, 2019);
11. Haussen, Diogo C, Brendan Eby, Alhamza R Al-Bayati, Jonathan A Grossberg, Gabriel Martins Rodrigues, Michael R Frankel, and Raul G Nogueira. A Comparative Analysis of 3MAX Aspiration versus 3 Mm Trevo Retriever for Distal Occlusion Thrombectomy in Acute Stroke. Journal of Neurointerventional Surgery 12, no. 3 (Mar. 1, 2020);
12. He G, Deng J, Lu H, et al. Thrombus enhancement sign on CT angiography is associated with the first pass effect of stent retrievers. J Neurointerv Surg 2022;None(None) doi: 10.1136/neurintsurg-2021-018447
13. Hesse, Amélie Carolina, Daniel Behme, André Kemmling, Antonia Zapf, Nils GroBe Hokamp, Isabelle Frischmuth, Ilko Maier, et al. Comparing Different Thrombectomy Techniques in Five Large-Volume Centers: A ‘real World’ Observational Study. Journal of Neurointerventional Surgery 10, no. 6 (Jun. 1, 2018);
14. Imahori, Taichiro, Yusuke Okamura, Junichi Sakata, Hiroyasu Shose, Akiyoshi Yokote, Kazushi Matsushima, Daisaku Matsui, et al. Stent Expansion and In-Stent Thrombus Sign in the Trevo Stent Retriever Predict Recanalization and Possible Etiology During Mechanical Thrombectomy: A Case Series of 50 Patients with Acute Middle Cerebral Artery Occlusion. World Neurosurg None, no. None (Dec. 28, 2018);
15. Imahori, Taichiro, Kazuhiro Tanaka, Atsushi Arai, Ryoji Shiomi, Daigo Fujiwara, Tatsuya Mori, Akiyoshi Yokote, et al. Mechanical Thrombectomy for Acute Ischemic Stroke Patients Aged 80 Years or Older. J Stroke Cerebrovasc Dis 26, no. 12 (Dec. 1, 2017);
16. Imahori, Taichiro, Kazuhiro Tanaka, Junji Koyama, Atsushi Arai, Ryoji Shiomi, Hirofumi Iwahashi, Akiyoshi Yokote, et al. Mechanical Thrombectomy Using the Trevo ProVue in 50 Consecutive Patients with Anterior Circulation Stroke: A Single-Center Experience after Approval of the Stent Retriever in Japan. Neurol Med Chir (Tokyo) 57, no. 3 (Mar. 15, 2017);
17. Jiang S, Fei A, Peng Y, et al. Predictors of Outcome and Hemorrhage in Patients Undergoing Endovascular Therapy with Solitaire Stent for Acute Ischemic Stroke. PLoS One 2015;10(12) doi: 10.1371/journal.pone.0144452
18. Jiang, Shao-wei, Hai-rong Wang, Ya Peng, Hui Sun, Miao Chen, Ai-hua Fei, and Shu-ming Pan. Mechanical Thrombectomy by Solitaire Stent for Treating Acute Ischemic Stroke: A Prospective Cohort Study. Int J Surg 28, no. None (Apr. 1, 2016);
19. Jiang L, Xia WQ, Huang H, et al. Mechanical Thrombectomy Outcome Predictors in Stroke Patients with M2 Occlusion: A Single-Center Retrospective Study. World Neurosurg 2019;127:e155-e61. doi: 10.1016/j.wneu.2019.03.013 [published Online First: 2019/03/16]
20. Jovin, Tudor G, Angel Chamorro, Erik Cobo, María A de Miquel, Carlos A Molina, Alex Rovira, Luis San Román, et al. Thrombectomy within 8 Hours after Symptom Onset in Ischemic Stroke. N Engl J Med 372, no. 24 (Jun. 11, 2015);
21. Kabbasch C, Mpotsaris A, Chang D-H, et al. Mechanical thrombectomy with the Trevo ProVue device in ischemic stroke patients: does improved visibility translate into a clinical benefit? J Neurointerv Surg 2016;8(8) doi: 10.1136/neurintsurg-2015-011861
22. Kabbasch, C, A Mpotsaris, T Liebig, M Söderman, M Holtmannspötter, M Cronqvist, J Thornton, V Mendes Pereira, and T Andersson. First-In-Man Procedural Experience with the Novel EmboTrap® Revascularization Device for the Treatment of Ischemic Stroke-A European Multicenter Series. Clinical Neuroradiology 26, no. 2 (Jun. 1, 2016);
23. Kaesmacher J, Chaloulos-Iakovidis P, Panos L, et al. Clinical effect of successful reperfusion in patients presenting with NIHSS < 8: data from the BEYOND-SWIFT registry. J Neurol 2019;266(3) doi: 10.1007/s00415-018-09172-1
24. Kammerer, S, R du Mesnil de Rochemont, M Wagner, S -J You, S Tritt, M Mueller-Eschner, F C Keil, A Lauer, and J Berkefeld. Efficacy of Mechanical Thrombectomy Using Stent Retriever and Balloon-Guiding Catheter. Cardiovascular and Interventional Radiology 41, no. 5 (May 1, 2018);
25. Kühn AL, Wakhloo AK, Lozano JD, et al. Two-year single-center experience with the ‘Baby Trevo’ stent retriever for mechanical thrombectomy in acute ischemic stroke. J Neurointerv Surg 2017;9(6) doi: 10.1136/neurintsurg-2016-012454
26. Lapergue B, Blanc R, Guedin P, et al. A Direct Aspiration, First Pass Technique (ADAPT) versus Stent Retrievers for Acute Stroke Therapy: An Observational Comparative Study. AJNR Am J Neuroradiol 2016;37(10) doi: 10.3174/ajnr.a4840
27. Li Z, Chu Z, Zhao S, et al. Severe Stroke Patients With Left-Sided Occlusion of the Proximal Anterior Circulation Benefit More From Thrombectomy. Front Neurol 2019;10(None) doi: 10.3389/fneur.2019.00551
28. Liang, Conrad W, Harjyot J Toor, Evelin Duran Martinez, Sunil A Sheth, Kuo Chao, Lei Feng, Mazen Noufal, Binh V Nguyen, Pankaj J Mowji, and Navdeep Sangha. First Pass Recanalization Rates of Solitaire vs Trevo vs Primary Aspiration: The Kaiser Southern California Experience. The Permanente Journal 25, no. None (Dec. 1, 2020);
29. Mattle HP, Carl S, Mairsil C, et al. Analysis of revascularisation in ischaemic stroke with EmboTrap (ARISE I study) and meta-analysis of thrombectomy. Interventional Neuroradiology 2019;25(3) doi: 10.1177/1591019918817406
30. Mokin M, Morr S, Natarajan SK, et al. Thrombus density predicts successful recanalization with Solitaire stent retriever thrombectomy in acute ischemic stroke. J Neurointerv Surg 2015;7(2) doi: 10.1136/neurintsurg-2013-011017
31. Mokin, Maxim, Christopher T Primiani, Alicia C Castonguay, Raul G Nogueira, Diogo C Haussen, Joey D English, Sudhakar R Satti, et al. First Pass Effect in Patients Treated With the Trevo Stent-Retriever: A TRACK Registry Study Analysis. Frontiers in Neurology 11, no. None (Jan. 1, 2020);
32. Nogueira, Raul G, Ashutosh P Jadhav, Diogo C Haussen, Alain Bonafe, Ronald F Budzik, Parita Bhuva, Dileep R Yavagal, et al. Thrombectomy 6 to 24 Hours after Stroke with a Mismatch between Deficit and Infarct. N Engl J Med 378, no. 1 (Jan. 4, 2018);
33. Oliver, Marion John, Emily Brereton, Muhib A Khan, Alan Davis, and Justin Singer. Trevo 6 × 25 mm vs. 4 × 30 mm in Mechanical Thrombectomy of Ml LVO. Front Neurol 12, no. None (Jan. 1, 2021);
34. Pfaff, Johannes, Stefan Rohde, Tobias Engelhorn, Arnd Doerfler, Martin Bendszus, and Markus Alfred Möhlenbruch. Mechanical Thrombectomy Using the New SolitaireTM Platinum Stent-Retriever : Reperfusion Results, Complication Rates and Early Neurological Outcome. Clin Neuroradiol 29, no. 2 (Jun. 1, 2019);
35. Pu, Yuan. Comparison of Different Types of Endovascular Mechanical Embolectomy in Acute Ischemic Stroke. Rev Assoc Med Bras (1992) 65, no. 3 (Mar. 1, 2019);
36. Sang, Hong-Fei, Cong-Guo Yin, Wen-Qing Xia, Huan Huang, Ke-Qin Liu, Tian-Wen Chen, Xiao-Li Si, and Lin Jiang. Mechanical Thrombectomy Using Solitaire in Acute Ischemic Stroke Patients with Vertebrobasilar Occlusion: A Prospective Observational Study. World Neurosurg 128, no. None (Aug. 1, 2019);
37. Saver JL, Goyal M, Bonafe A, et al. Stent-retriever thrombectomy after intravenous t-PA vs. t-PA alone in stroke. N Engl J Med 2015;372(24) doi: 10.1056/nejmoal415061
38. Slezak A, Kurmann R, Oppliger L, et al. Impact of Anesthesia on the Outcome of Acute Ischemic Stroke after Endovascular Treatment with the Solitaire Stent Retriever. AJNR Am J Neuroradiol 2017;38(7) doi: 10.3174/ajnr.a5183
39. Srivatsan, A, V M Srinivasan, R M Starke, E C Peterson, D R Yavagal, A E Hassan, A Alawieh, et al. Early Postmarket Results with EmboTrap II Stent Retriever for Mechanical Thrombectomy: A Multicenter Experience. AJNR. American Journal of Neuroradiology None, no. None (Mar. 11, 2021);
40. Sztajzel RF, Muller H, Sekoranja L, et al. Strokes in the anterior circulation: comparison between bridging and intravenous thrombolysis. Acta Neurol Scand 2015;131(5) doi: 10.1111/ane.12338
41. Valente, Iacopo, Sergio Nappini, Leonardo Renieri, Alessandro Pedicelli, Emilio Lozupone, Cesare Colosimo, Salvatore Mangiafico, and Nicola Limbucci. Initial Experience with the Novel EmboTrap II Clot-Retrieving Device for the Treatment of Ischaemic Stroke. Interventional Neuroradiology: Journal of Peritherapeutic Neuroradiology, Surgical Procedures and Related Neurosciences 25, no. 3 (Jun. 1, 2019);
42. Wiącek, Marcin, Rafał Kaczorowski, Jarosław Homa, Edward Filip, Janusz Darocha, Daniel Dudek, Wiesław Guz, and Halina Bartosik-Psujek. Single-Center Experience of Stent Retriever Thrombectomy in Acute Ischemic Stroke. Neurol Neurochir Pol 51, no. 1 (Jan. 1, 2017);
43. Yang, D, Y Hao, W Zi, H Wang, D Zheng, H Li, M Tu, et al. Effect of Retrievable Stent Size on Endovascular Treatment of Acute Ischemic Stroke: A Multicenter Study. AJNR Am J Neuroradiol 38, no. 8 (Aug. 1, 2017);
44. Yang, Xingxiu, Xiaohui Jia, Hua Ren, and Hongxing Zhang. The Short- and Long-Term Efficacies of Endovascular Interventions for the Treatment of Acute Ischemic Stroke Patients. Am J Transl Res 13, no. 5 (Jan. 1, 2021);
45. Yi, Ho Jun, Dong Hoon Lee, and Sang Uk Kim. Effectiveness of Trevo Stent Retriever in Acute Ischemic Stroke: Comparison with Solitaire Stent. Medicine 97, no. 20 (May 1,2018);
46. Yi, Ting-Yu, Wen-Huo Chen, Yan-Min Wu, Mei-Fang Zhang, Ding-Lai Lin, and Xiao-Hui Lin. Adjuvant Intra-Arterial Rt-PA Injection at the Initially Deployed Solitaire Stent Enhances the Efficacy of Mechanical Thrombectomy in Acute Ischemic Stroke. J Neurol Sci 386, no. None (Mar. 15, 2018);
47. Zaidat, Osama O, Hormozd Bozorgchami, Marc Ribó, Jeffrey L Saver, Heinrich P Mattle, René Chapot, Ana Paula Narata, et al. Primary Results of the Multicenter ARISE II Study (Analysis of Revascularization in Ischemic Stroke With EmboTrap). Stroke 49, no. 5 (May 1, 2018);
48. Zaidat, Osama O, Alicia C Castonguay, Rishi Gupta, Chung-Huan J Sun, Coleman Martin, William E Holloway, Nils Mueller-Kronast, et al. North American Solitaire Stent Retriever Acute Stroke Registry: Post-Marketing Revascularization and Clinical Outcome Results. J Neurointerv Surg 10, no. Suppl 1 (Jul. 1, 2018);
49. Zaidat, Osama O, Nils H Mueller-Kronast, Ameer E Hassan, Diogo C Haussen, Ashutosh P Jadhav, Michael T Froehler, Reza Jahan, et al. Impact of Balloon Guide Catheter Use on Clinical and Angiographic Outcomes in the STRATIS Stroke Thrombectomy Registry. Stroke 50, no. 3 (Mar. 1, 2019);
50. Zhou T-F, Zhu L-F, Li T-X, et al. Application of retrievable Solitaire AB stents in the endovascular treatment of acute ischemic stroke. J Interv Med 2019;1(2) doi: 10.19779/j.cnki.2096-3602.2018.02.03; and
51. Zhou T, Li T, Zhu L, et al. Comparing the efficacy and safety of the Skyflow device with those of the Solitaire FR stent in patients with acute ischemic stroke: a prospective, multicenter, randomized, non-inferiority clinical trial. J Neurointerv Surg 2021;None(None) doi: 10.1136/neurintsurg-2021-018117,

Each of the fifty-one corresponding studies listed above are hereby incorporated by reference in their entirety as if set forth verbatim herein. It is understood that data is presented herein for purposes of illustration and should not be construed as limiting the scope of the disclosed technology in any way or excluding any alternative or additional embodiments.

A primary objective of the study of this disclosure was to compare real-world primary outcomes among patients undergoing mechanical thrombectomy procedures for treatment of acute ischemic stroke with revascularization or clot retrieval device 200, which can include the EmboTrap retrieval device (EmboTrap, CERENOVUS, Miami, FL, USA) with comparative device 1 (Trevo, STRYKER, Kalamazoo, MI, USA) and comparative device 2 (Solitaire, MEDTRONIC, Dublin, Ireland). Device 200 can include an open outer cage designed for clot capture and a closed inner channel designed for clot stabilization, as shown in U.S. Pat. Nos. 8,777,976; 8,852,205; 9,402,707; 9,445,829; and 9,642,639.

Initial prospective, multi-center studies using device 200 reported high reperfusion rates (mTICI≥2b within 3 passes of device 200 without rescue) of 75.0% and 80.2%, respectively. See Zaidat OO, Bozorgchami H, Ribo M, et al. Primary Results of the Multicenter ARISE II Study (Analysis of Revascularization in Ischemic Stroke With EmboTrap). Stroke 2018;49:1107-15. These initial prospective studies had prespecified patient inclusion and exclusion criteria, and thus patient outcomes may differ from real-world use of device 200.

Though previous randomized clinical trials have demonstrated that mechanical thrombectomy (MT) using stent retrievers is a safe and effective treatment for acute ischemic stroke (AIS) due to large vessel occlusion (LVO), these studies have been limited to using one device or the other. These previous studies did not examine recanalization, functional and safety outcomes after treatment with either device 200, comparative device 1 or comparative device 2 in patients with acute ischemic stroke from a retrospective analysis or meta-analysis perspective.

To resolve the limitation, a PRISMA compliant systematic literature review utilizing the Nested Knowledge AutoLit platform was used. See Page, M.J. et al. The PRISMA 2020 statement: An updated guideline for reporting systematic reviews. PLOS Medicine 18 (3) (2021) el003583. All study variables are summarized as the mean and standard deviation for continuous variables and the frequency and percentage for categorical variables. Means and proportions were logarithmically transformed prior to fitting models. After pooling, pooled estimates were back-transformed to aid in interpretation. Haldane-Anscombe correction was conditionally applied to correct for zero-cell counts. See Haldane, J. The mean and variance of the moments of chi square, when used as a test of homogeneity, when expectations are small. Biometrika 29, 133 - 143 (1940); see also Anscombe, F. On estimating binomial response relations. Biometrika 43, 461 - 464 (1956). The DerSimonian-Laird procedure for fitting the random effects model for meta-analysis. See DerSimonian R., Laird N. Meta-analysis in clinical trials. Control Clin Trials 7, 177-188 (1986). Corresponding 95% confidence interval (“CI”) was computed using the Jackson method. See Jackson, D. Confidence intervals for the between-study variance in random effects meta-analysis using generalised Cochran heterogeneity statistics. Res Synth Methods 4, 220-229 (2013). Methods described in McGrath et al were used to aggregate median mRS scores. See McGrath, S. et al. One-sample aggregate data meta-analysis of medians. Stat Med 38, 969-984 (2019). I² values based on Higgins et al., were used to estimate the percentage of statistical inconsistency unattributable to sample error. See Higgins J.P. et al., Measuring inconsistency in meta-analyses. BMJ. 327, 557-560 (2003); see also Mirza, M. et al. Systematic review and analysis of pre-clinical side-by-side comparisons of EmboTrap versus Solitaire performance. In ESMINT (2021).

For all studies, patient characteristics including age, use of intravenous thrombolysis (IVT) with tissue plasminogen activator (tPA), use of balloon guide catheter (BGC), preoperative NIHSS, ASPECTS score, and stroke location were collected. In addition, outcome data including 90-day mRS, 90-day mortality, embolization to new territory (ENT), symptomatic intracranial hemorrhage (sICH), final TICI or mTICI, and first pass recanalization (FPR) was extracted.

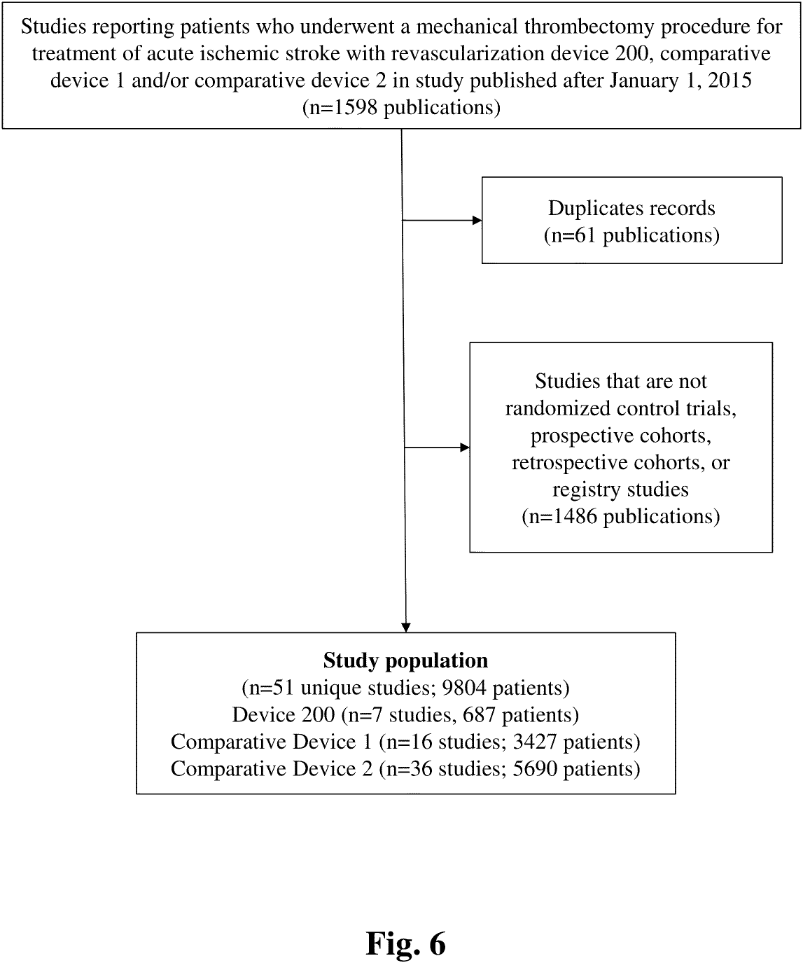

The study cohort attrition process is shown in FIG. 6. Studies reporting patients who underwent a mechanical thrombectomy procedure for treatment of acute ischemic stroke (International Classification of Disease, 9th edition, clinical modification codes for Acute Ischemic Stroke (ICD-9-CM): 433.xx; 434.xx; 436.xx, 437.xx, 438.xx and equivalent ICD-10-CM code) with revascularization device 200, comparative device 1, and/or comparative device 2 in studies published after Jan. 1, 2015 were initially identified through Nested Knowledge AutoLit platforms and PubMed/MEDLINE databases. The platforms and databases were systemically searched for devices of interest and outcomes of interest relating to acute ischemic stroke and thrombectomy. Eligible studies included randomized control trials, prospective cohorts, retrospective cohorts, and registry studies. Studies that used one of device 200, comparative device 1, or comparative device 2 for treatment of acute ischemic stroke were included. Studies were excluded if they: 1) did not use a device of interest, 2) did not report any outcomes of interest, 3) only reported combination treatment (SR used with an aspiration device), 4) did not separate patient outcomes by technique or device used, 5) had <25 patients, 6) did not relate to AIS, 7) were the wrong study type (in vivo/in vitro study, symposium/conference, case report, qualitative review, letter of correspondence, in silico study/mathematical model, guideline article, technical note, editorial/opinion, cost-effectiveness study, survey study, economic study without angiographic or clinical outcomes, meta-analysis/systematic review, secondary analysis, protocol, or interim analysis), 8) did not report patients treated with MT, 9) reported a biased subset of the stroke population (such as reporting only stroke locations known to be associated with worse outcomes), or 10) full text was unavailable. Studies were also excluded if they were non-English articles, published before Jan. 1, 2015, or had less than 25 patients.

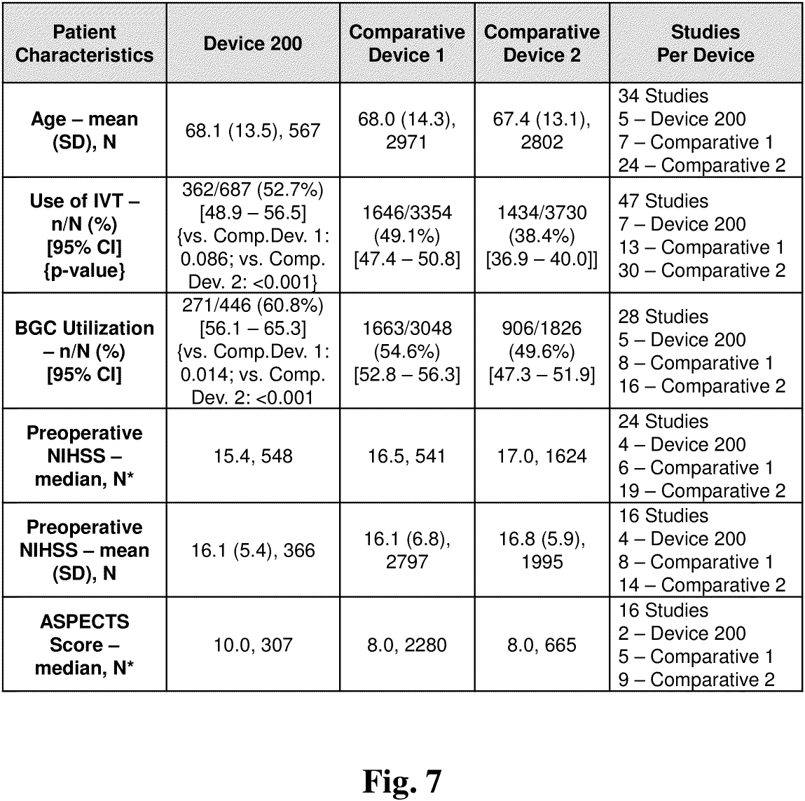

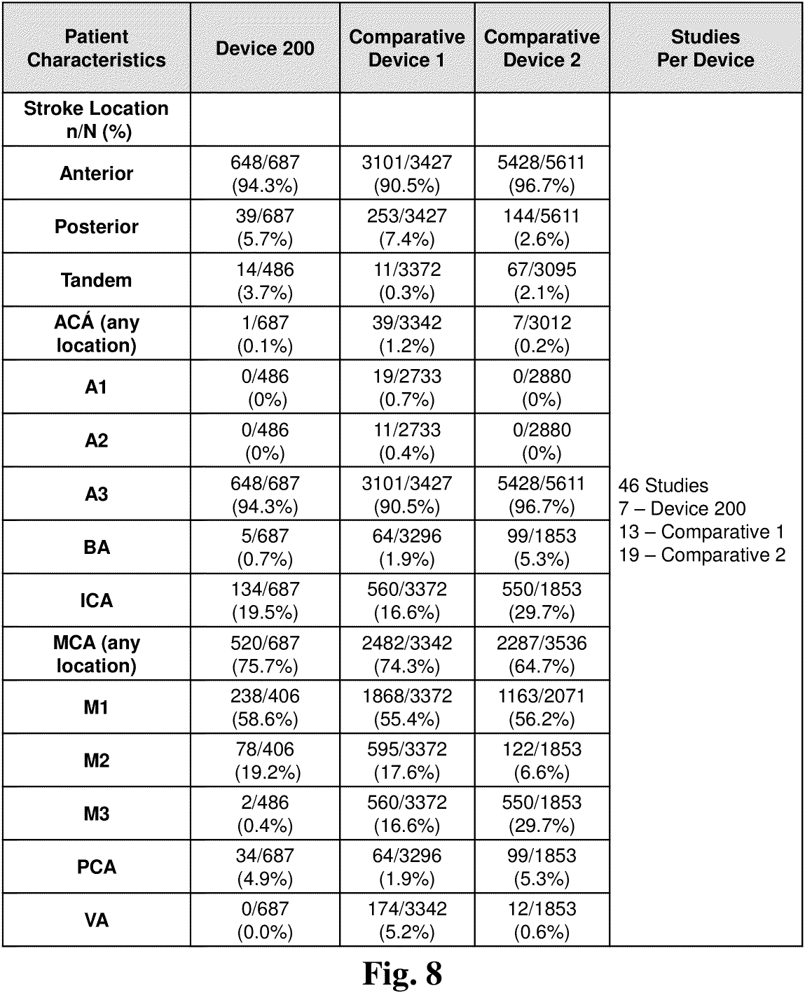

FIGS. 7 and 8 show tables summarizing background characteristics of patients treated with either device 200, comparative device 1, or comparative device 2. Of the 9,804 patients identified in the 51 studies, 687 patients from a total of 7 studies were treated with device 200, 3,427 patients from a total of 16 studies were treated with comparative device 1, and 5,690 patients from a total of 36 studies were treated with comparative device 2. Eleven studies reported core-lab adjudicated angiographic results, including two studies with device 200, three studies with comparative device 1, and 11 studies with comparative device 2. Some studies did not report certain patient characteristics such as pre-operative procedures, NIHSS, or mean age. For instance, only 5 out of 7 studies provided sufficient data of patient age for patients treated with device 200 for acute ischemic stroke.

The mean age of patients in device 200, comparative device 1 and comparative device 2 groups were 69.2 years, 68.7 years, and 66.4 years, respectively. The overall baseline mean NIHSS scores were 16.3 for all devices. The mean ASPECTS scores were 8.8 for device 200, 9.5 for comparative device 2, but not reported in underlying studies for comparative device 1. For device 200, 648/687 (94.3%) of occlusions were in the anterior circulation and 39/687 (5.7%) were in the posterior circulation, while for comparative device 1, 3101/3427 (90.5%) of occlusions were anterior and 253/3427 (7.4%) were posterior, and for comparative device 2, 5428/5611 (96.7%) were anterior and 144/5611 (2.6%) were posterior.

Balloon-guided catheter (“BGC”) use patterns were reported in 5/7 (71.4%) device 200 study arms, in 8/14 (57.1%) study arms for comparative device 1, and in 16/34 (47.1%) study arms for comparative device 2. Among studies that reported BGC use, rates were 60.8% (271/446; 95% CI 56.1-65.3%) for device 200, 54.6% (1663/3048; 95% CI 52.8-56.3%) for comparative device 1, and 49.6% (906/1826; 95% CI 47.3-51.9%) for comparative device 2. The device 200 group had a statistically significant higher rate of BGC use compared to both the comparative 1 group (p=0.014) and the comparative 2 group (p<0.001). Of study arms that reported IV-tPA use (7/7 [100%] of device 200 study arms, 13/14 [92.9%] for comparative device 1, and 30/32 [88.2%] for comparative device 2), rates were 52.7% (362/687; 95% CI 48.9-56.5%) for device 200, 49.1% (1646/3354; 95% CI 47.4-50.8%) for Comparative device 1, and 38.4% (1434/3730; 95% CI 36.9-40.0%) for Comparative device 2. The device 200 group had a statistically significant higher rate of IVT use compared to the Comparative device 2 group (p<0.001) and trended to have a higher rate compared to the Comparative device 1 group (p=0.086). Additionally, the Comparative device 1 group had a statistically significant higher rate of IVT use compared to the Comparative device 2 group (p<0.001). See FIGS. 6 and 7 for pooled summary statistics of baseline characteristics and Appendix 1 for a complete list of study and patient baseline characteristics at the study level.

### Complete Recanalization on First Pass (FPR mTICI ≥2c)

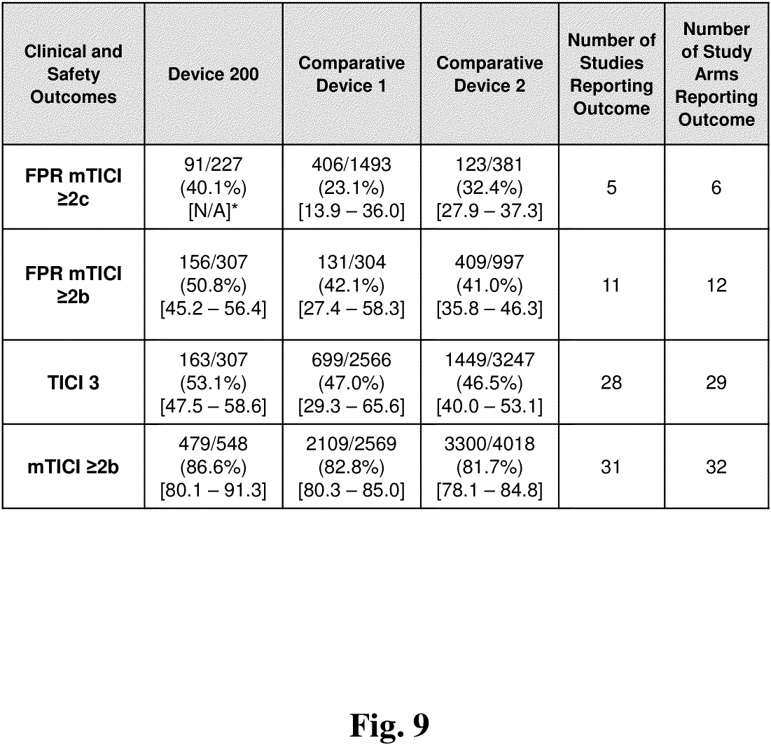

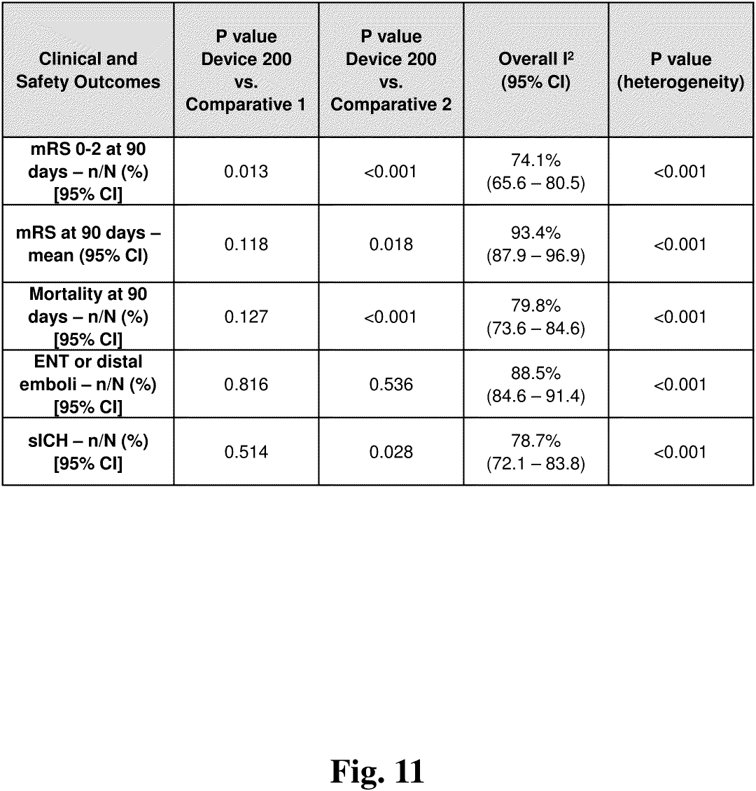

Among the 5 studies with sufficient data, pooled rates of FPR mTICI ≥2c were 40.1% (95% CI not available) for device 200, 23.1% (95% CI 13.9-36.0) for comparative device 1, and 32.4% (95% CI 27.9-37.3) for comparative device 2 (FIGS. 9-11). A formal statistical comparison between device 200 and other devices was not performed as only one device 200 study reported this outcome, hence between-study variance could not be estimated. Pooled rates of FPR mTICI ≥2c did not differ significantly between comparative device 2 and comparative device 1 (p=0.220; Appendix 1). Outlier and influence analyses were not performed for FPR mTICI ≥2c and subsequent recanalization outcomes due to limited number of studies reporting data.

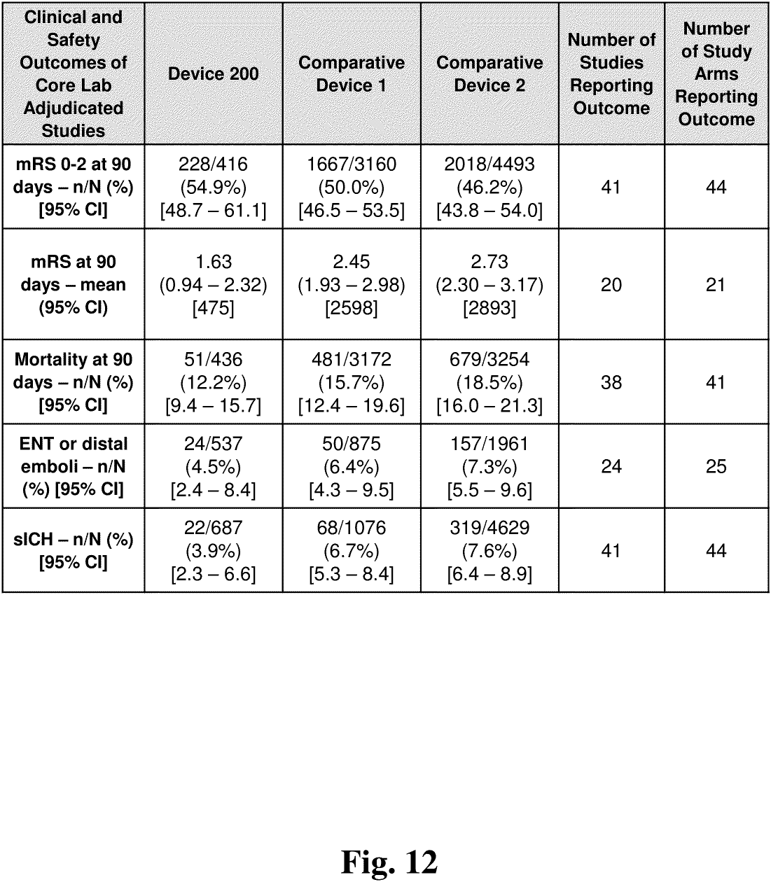

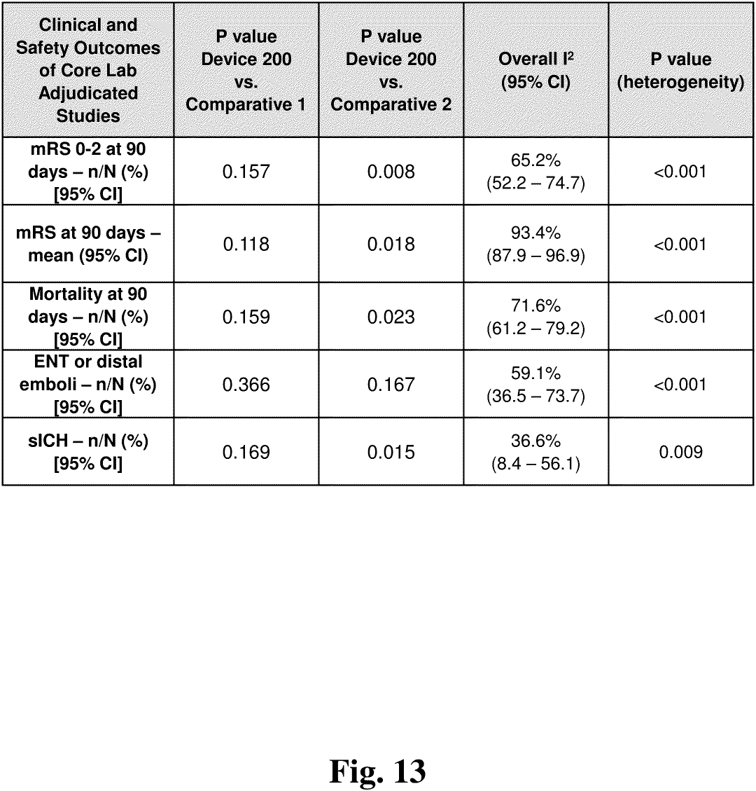

FPR mTICI ≥2c results from studies that used an independent core lab to adjudicate recanalization outcomes are presented in FIGS. 12 and 13. Rates among core lab adjudicated studies were 40.1% for Device 200, 27.6% for comparative device 1, and 25.8% for comparative device 2.

### Successful Recanalization on First Pass (FPR mTICI ≥2b)

Among the 11 studies with sufficient data, pooled rates of FPR mTICI ≥2b were 50.8% (95% CI 45.2-56.4) for device 200, 42.1% (95% CI 27.4-58.3%) for comparative device 1, and 41.0% (95% CI 35.8-46.3) for comparative device 2; these rates did not differ significantly (p=0.430; FIGS. 9-11 and Appendix 1).

Rates of FPR mTICI ≥2b among core lab adjudicated studies were 51.5% for device 200, 32.7% for comparative device 1, and 35.8% for comparative device 2; no formal statistical analyses were performed to determine significance due to insufficient number of studies (FIGS. 12 and 13).

Rates of FPR mTICI ≥2b among prospective studies were 51.5% for device 200, 36.3% (95% CI 26.3-47.8) for comparative device 1, and 47.0% (95% CI 42.0-52.0) for comparative device 2. No formal pairwise comparisons relative to the device 200 group were performed due to insufficient number of studies. Rates of FPR mTICI ≥2b among prospective studies trended higher in the comparative device 1 group compared to the comparative device 2 group (p=0.055; See Appendix 1).

### Final Complete Recanalization (TICI 3)

Among all 28 studies with sufficient data, pooled rates of TICI 3 did not differ significantly (p=0.879, Appendix 1) between device 200 (53.1%, 95% CI 47.5-58.6), comparative device 1 (47.0%, 95% CI 29.3-65.6), and comparative device 2 (46.5%, 95% CI 40.0-53.1) (FIGS. 9-11). Reported rates of TICI 3 varied substantially between studies, with an estimated 95.7% (95% CI 94.6-96.5) of the variability attributable to heterogeneity rather than sampling error.

When core-lab adjudicated studies were analyzed, rates of TICI 3 trended lower for comparative device 1 (14.1%) and comparative device 2 (35.4%) compared to device 200 (52.0%), but a formal statistical comparison of treatment groups was not performed due to limited study data (FIGS. 12 and 13).

Evaluating the prospective studies only, the rates of TICI3 were 52.0% for device 200, 28.8% (95% CI 6.1-71.6) for comparative device 1, and 44.9% (95% CI 28.5-62.6) for comparative device 2. No formal pairwise comparisons relative to the device 200 group were performed due to insufficient number of studies.

### Final Successful Recanalization (mTICI ≥2b)

Among all the 31 studies with sufficient data, pooled rates of mTICI ≥2b did not differ according to the omnibus test of subgroup differences (p=0.202; Appendix 1) and were 86.6% (95% CI 80.1-91.3%) for device 200, 82.8% (95% CI 80.3-85.0%) for comparative device 1, and 81.7% (95% CI 78.1-84.8%) for comparative device 2 (FIGS. 9-11).

Among the 11 core lab adjudicated studies with sufficient data, although not statistically significant, pooled rates of final mTICI ≥2b for device 200 trended higher in comparison to comparative device 2 (89.0%, 95% CI78.5-94.7 vs. 80.0%, 95% CI73.4-85.4; p=0.052) and comparative device 1 (81.8%, 95% CI 79.9-83.5; p=0.224) (see FIGS. 12 and 13).

Among the 13 prospective only studies with sufficient data, pooled rates of mTICI≥2b did not differ significantly between subgroups according to the omnibus test of subgroup differences (p=0.849; Appendix 1) and were 88.1% (95% CI 80.5-93.0%) for device 200, 86.4% (95% CI 77.7-92.1%) for comparative device 1, and 85.7% (95% CI 78.9-90.5%) for comparative device 2 (Appendix 1).

### Embolization to New Territory (ENT) or Distal Emboli

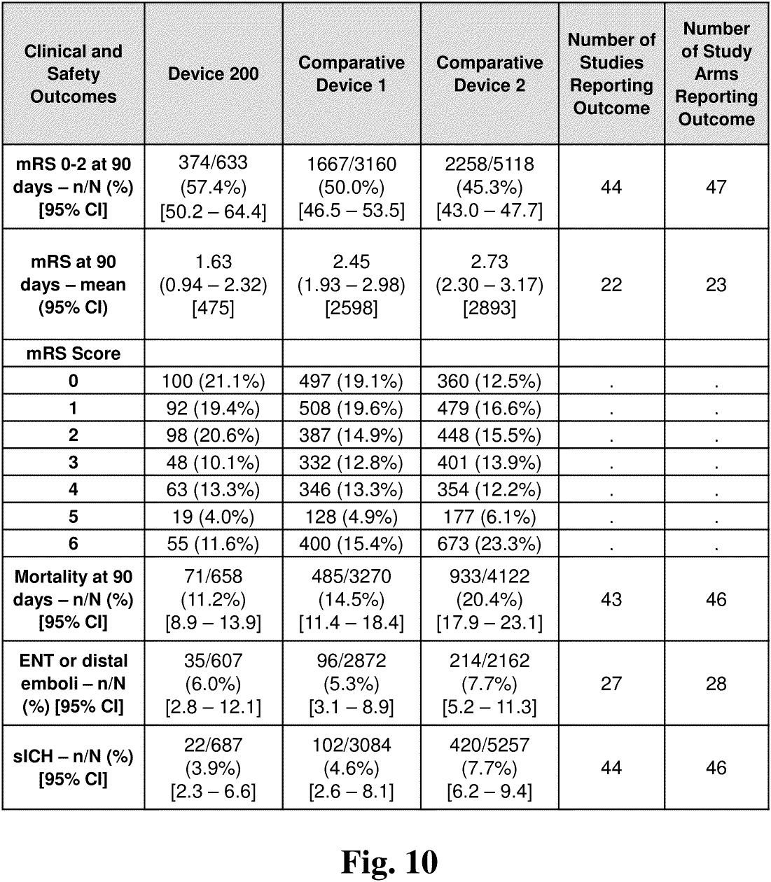

Among all the 27 studies with sufficient data, pooled rates of emboli (reflecting total reporting of ENT and distal emboli) did not differ significantly according to the omnibus test of subgroup differences (p=0.518; Supplementary FIG. 10) and were 6.0% (95% CI 2.8-12.1) for device 200, 5.3% (95% CI 3.1-8.9) for comparative device 1, and 7.7% (95% CI5.2-11.3) for comparative device 2 (FIGS. 9-11).

After excluding 3 outlier findings, 24 studies had sufficient data for comparisons of ENT rates. Pooled rates of ENT and distal emboli did not differ significantly between device 200 (4.5%, 95% CI 2.4-8.4), comparative device 1 (6.4%, 95% CI 4.3-9.5), and comparative device 2 (7.3%, 95% CI 5.5-9.6). Overall, results did not substantially differ compared to the complete-case analysis, but directionality of outcome comparisons changed, with results favoring comparative device 1 before removing outliers and results favoring the device 200 group after outlier removal. Relative to the complete-case analysis, statistical heterogeneity was reduced by 29.9% (I2=88.5%, 95% CI 84.6-91.4 vs. I²=58.6%, 95% CI 33.0-74.5). Outcome comparisons after outlier removal for ENT/distal emboli and other safety and clinical outcomes are presented in Appendix 1.

Among the 11 prospective only studies with sufficient data, pooled rates of ENT did not differ significantly between subgroups according to the omnibus test of subgroup differences (p=0.426; Appendix 1) and were 3.9% (95% CI 1.2-12.1%) for device 200, 2.8% (95% CI 1.3-5.8%) for comparative device 1, and 5.4% (95% CI 2.8-10.0%) for comparative device 2 (Appendix 1), with a smaller difference but similar direction to the complete-case analysis.

### Symptomatic Intracranial Hemorrhage (sICH)

Among all the 44 studies with sufficient data, pooled rates of sICH for comparative device 2 (7.7%, 95% CI 6.2-9.4) were significantly higher compared to both device 200 (3.9%, 95% CI 2.3-6.6; p=0.028) and comparative device 1 (4.6%, 95% CI 2.6-8.1; p=0.049) (FIGS. 9-11).

After excluding two outlier studies, 42 studies had sufficient data for comparisons of sICH rates. Device 200 (3.9%, 95% CI 2.3-6.6) maintained significantly lower pooled rate of sICH compared to comparative device 2 (7.6%, 95% CI 6.4-8.9%; p=0.015).; pooled rates of sICH for comparative device 1 were 6.7% (95% CI 5.3-8.4) and did not differ significantly from the other devices (Appendix 1). Overall, outcome comparisons differed with respect to the complete-case analysis and the observed statistical heterogeneity was reduced by 42.1% (I2=78.7%, 95% CI 72.1-83.8 vs. I2=36.6%, 95% CI 8.4-56.1).

Among the 19 prospective only studies with sufficient data, pooled rates of sICH for comparative device 2 (7.3%, 95% CI 5.0-10.7) were significantly higher compared to device 200 (2.2%, 95% CI 0.7-7.2; p=0.044) and comparative device 1 (1.7%, 95% CI 1.2-2.3; p=0.004) (Appendix 1).

### Modified Rankin Scale (mRS) Score at 90 Days

Among all the 44 studies with sufficient data, pooled rates of mRS 0-2 for device 200 (57.4%,95% CI 50.2-64.4) were significantly higher compared to comparative device 1 (50.0%, 95% CI 46.5-53.5; p=0.013) and comparative device 2 (45.3%, 95% CI 43.0-47.7; p<0.001) (FIGS. 9-11 and Appendix 1).

After excluding four outlier studies, 41 studies had sufficient data for comparisons of mRS 0-2 at 90 days. Patients treated with device 200 had a significantly higher pooled rate of mRS 0-2 at 90 days (54.9%, 95% CI 48.7-61.1) compared to comparative device 2 (46.2%, 95% CI 43.8-54.0; p=0.008); pooled rates for comparative device 1 (50.0%, 95% CI 46.5-53.5) did not differ significantly from the other devices at the α=0.05 level, but the directionality and magnitude of effect estimates were similar to the complete-case analysis (Appendix 1). Compared to the complete-case analysis, there was a relatively small reduction in statistical heterogeneity (I2=78.7%, 95% CI 72.1-83.8 vs. I2=65.2%, 95% CI 52.2-74.7).

Among the 17 prospective studies with sufficient data, pooled rates of mRS 0-2 for comparative device 2 (43.4%, 95% CI 40.1-46.8) were significantly lower compared to device 200 (56.8%, 95% CI 44.7-68.1; p<0.001) and comparative device 1 (55.1%, 95% CI 53.0-57.3; p=0.029) (Appendix 1). No significant difference in mRS 0-2 rates was observed between device 200 and comparative device 1 (p=0.398).

Among the 22 studies with sufficient data to compare ordinal mRS scores at 90 days, pooled median scores were lower for patients treated with device 200 (1.63, 95% CI 0.94-2.23) compared to comparative device 2 (2.73, 95% CI 2.30-3.17; p=0.018); the pooled median mRS score for patients treated with comparative device 1 was 2.45 (95% CI 1.93-2.98) and did not differ significantly from the other devices (p=0.118 vs. device 200, p=0.441 vs. comparative device 2; FIGS. 9-11). A table of combined ordinal mRS scores across treatment groups is shown in Appendix 1.

Among the 10 prospective studies with sufficient data to compare ordinal mRS scores at 90 days, pooled median scores trended higher for patients treated with comparative device 2 (2.82, 95% CI 2.24-3.40) compared to device 200 (1.50, 95% CI 0.52-2.48; p=0.079) and comparative device 1 2.00 (95% CI 1.93-2.06; p=0.111), but differences between groups were not statistically significant (Appendix 1).

### Mortality at 90 Days

Among the 43 studies with sufficient data, the pooled rate of mortality at 90 days for comparative device 2 (20.4%, 95% CI 17.9-23.1) was significantly higher compared to device 200 (11.2%, 95% CI 8.9-13.9; p<0.001) and comparative device 1 (14.5%, 95% CI 11.4-18.4; p=0.018). There was no statistically significant difference between device 200 and comparative device 1 with respect to mortality (p=0.127) (FIGS. 9-11).

After excluding five outlier studies, 38 studies had sufficient data for comparisons of 90-day mortality rates. Device 200 (12.2%, 95% CI 9.4-15.7%) had a significantly lower pooled 90-day mortality rate compared to comparative device 2 (18.5%, 95% CI 16.0-21.3%; p=0.023); pooled rates for comparative device 1 (15.7%, 95% CI 12.4-19.6%) did not differ significantly from the other devices (Appendix 1). Overall, the directionality and magnitude of effect estimates were similar to the complete-case analysis, but there was a relatively small reduction in statistical heterogeneity (I2=79.8%, 95% CI 73.6-84.6 vs. I2=71.6%, 95% CI 61.2-79.2).

Among the 15 prospective studies with sufficient data, pooled rates of mortality at 90 days for comparative device 2 (21.9%, 95% CI 19.0-25.2) were significantly higher compared to both device 200 (11.0%, 95% CI7.5-15.9; p<0.001) and comparative device 1 (13.6%, 95% CI 8.8-20.3; p<0.001) (Appendix 1). No significant difference in mortality at 90 days was found between device 200 and comparative device 1 (p=0.215).

Device 200 can provide significantly lower rates of sICH and 90-day mortality compared to comparative device 2 and significantly higher rates of mRS 0-2 at 90-days compared to both comparative device 2 and comparative device 1. When comparative device 1 and comparative device 2 were compared, comparative device 1 demonstrated significantly lower rates of sICH and mortality, though improvements in sICH were not robust following outlier analysis. Final recanalization outcomes (successful recanalization/mTICI ≥2b and complete recanalization/TICI 3) for device 200 trended numerically higher compared to comparative device 2 and comparative device 1. Similarly, successful FPR and complete FPR for device 200 trended higher compared to comparative device 2 and comparative device 1, however due to a dearth of studies reporting FPR, a formal statistical analysis, including outlier and influence analysis could not be performed. The dearth of FPR data in stroke studies is expected as FPR was first defined and reported in 2018 by Zaidat et al.

While these findings are explorative and informative, they must be seen in the context of a potentially heterogenous patient population. Mean age, NIHSS, and ASPECTS, where reported, were relatively consistent across underlying different SR studies. In terms of stroke location, posterior strokes have been shown to have consistently worse clinical outcomes. The device with the lowest proportion of posterior strokes, comparative device 2, did not have improved clinical outcomes over the other devices; this does not indicate that stroke location had no impact on outcomes, but rather that a greater proportion of anterior strokes did not lead to improved clinical performance for comparative device 2.

Furthermore, differences in the procedural workflow beyond SR device choice may contribute to differences in outcomes. For instance, the fact that pooled rates of BGC use and IV-tPA use were higher for device 200 and comparative device 1 compared to comparative device 2 may contribute to more favorable outcomes. While variation in procedural practices were narrowed as much as possible—for instance, techniques combining SRs with aspiration catheters were excluded from the analysis to ensure that the use of adjunctive aspiration did not impact the head-to-head comparison of SRs—other differences in procedural practice, such as differences in the sizes and lengths of each device, and differences in operator experience or change in practices over the period of this meta-analysis, were impossible to control for in our meta-analysis.

The limitations in controlling for background and procedural variables, and the high heterogeneity in many of the comparisons in this meta-analysis, makes mitigating and finding the source of heterogeneity a priority. In addition to excluding studies based on potentially biasing population characteristics or procedural practices, an outlier analysis was performed to detect potential sources of heterogeneity that may disproportionately sway results in favor of any given treatment group. As its findings show, the heterogeneity was systematically lower in outlier-adjusted analysis, and both safety outcomes and functional outcomes had changes in significant findings after adjustment for outliers. This limitation could be addressed with further multi-arm studies of the devices in question. While this meta-analysis provides preliminary findings regarding the overall rate of emboli and sICH, variations in the reporting by underlying studies may have impacted the rate of both outcomes, and only a direct comparison using the same criteria with respect to all treated populations can adjust for these differences in procedural practice and in defining outcomes.

Another benefit of further RCT-level comparisons of the devices in question is the use of core lab adjudication of angiographic results. In this meta-analysis, no differences in performance were found when site-adjudicated data were analyzed alongside core, but when the core-adjudicated subset was analyzed, substantially lower rates of FPR mTICI ≥2b were found for comparative device 2 and comparative device 1, and the rate of TICI 3 for comparative device 1 fell by more than half. This reflects potential site-adjudication bias, aligning with previous findings that TICI ratings have been previously found to vary by observer, sometimes leading to inflated reported rates of success. The core lab subanalysis supports the continued use of core lab adjudication where possible, for the final reporting of any angiographic findings that may be subject to inaccuracy or bias in site-level assessment.

Device 200 was designed to improve clot engagement in thrombectomy across a range of clots with varying composition. While device 200 outperformed other SRs in the current study, to date, no head-to-head RCTs comparing SRs for use in MT for AIS have been completed. However, a recent pre-clinical systematic review to investigate the impact of device design on MT success found that device 200 performed significantly better than comparative device 2 (p<0.01), particularly for friable (p<0.05) and standard (p<0.05) clot types. In a previous meta-analysis of SR plus aspiration (combination technique), it was found that device 200 outperformed comparative device 2 and comparative device 1 when used with concomitant aspiration with respect to FPR mTICI 2b/3 and recanalization before rescue. It was also found that device 200 outperformed comparative device 2 and comparative device 1 when used with aspiration catheters with inner diameters ≥0.068″ but not when used with aspiration catheters <0.068″ and attributed this finding to device 200′s design/geometry differences. The combination techniques described herein suggest that design differences in device 200 may translate into meaningful advantages in achieving important safety and clinical outcomes.

These promising results must be balanced against the limited amount of prospective and large-population studies of device 200. Among the seven studies of device 200 included herein, two were prospective cohort studies, the Analysis of Revascularization in Ischemic Stroke with device 200 I and II (ARISE I and ARISE II). These initial studies reported high reperfusion rates (mTICI ≥2b)of 85.0% and 92.5%, respectively. As rigorous clinical trials, the ARISE studies may have included a less diverse patient and provider population or provided a higher level of clinical care and follow-up. However, real-world studies using device 200 have demonstrated similar results. Four European studies have been conducted since 2016; one reported a reperfusion rate of 95.0% (TICI ≥2b), and three documented rates of 84.6-95.7% (mTICI ≥2b). A US multi-center registry of patients treated with device 200, a device iteration that incorporates a double proximal marker for more precise stent placement and an increase from 3 to 5 outer cages, found a similarly high rate of successful reperfusion of 95.7% (TICI ≥2b). Similarly, the rates of sICH for patients treated with device 200 found in the clinical trials are within the range of findings from real-world device 200 studies. ARISE II found that 5.3% of trial patients experienced a sICH, while the rate in real-world studies ranged from 0.5% to 6.3%.

While it is noted statistically significant improved differences in the rate of sICH and mortality among patients treated with comparative device 1 compared to comparative device 2, these differences were no longer statistically significant after outlier studies were removed. In the case of sICH, Binning et al. found an sICH rate of 1.7% in a sample of 2008 patients treated with comparative device 1, which represented an outlier. Hence, this finding substantially shifted results toward a lower sICH pooled rate for comparative device 1. After removal of one outlier study, comparative device 1 lost its significant difference in sICH compared to comparative device 2, suggesting that the comparative device 1 results are not robust to the outlier. Among comparative device 2 studies, a study with an sICH rate of 16.1% was an outlier, perhaps due to the relatively high rate of difficult strokes included in the study sample. However, removal of this study did not have a large impact on comparative device 2 findings, suggesting that the pooled estimate for comparative device 2 is relatively robust to the outlier. Overall, after removal of these outliers reduced heterogeneity (I²) was observed and the estimated percentage of variability in effect estimates for sICH was much lower and revealed a more consistent pattern. In the same vein, the ARISE II clinical trial of device 200 was also found to be an influential outlier with respect to neurological outcome. The removal of ARISE II, and other outlier studies, from the analysis attenuated the difference in 90-day mRS 0-2 found between device 200 and other SRs. Although the differences in 90-day mRS 0-2 remained statistically significantly favorable for device 200 over comparative device 2, the difference compared to comparative device 1 was no longer statistically significant.

While it is not always possible to determine what aspect of a study or patient population contributed to unusually high or low rates of important outcomes, some potential factors include procedural or operator differences (i.e. use of BGCs), more difficult patient populations (i.e., higher proportion of ICA vs. MCA strokes which may be more difficult to treat), differences in study design factors (such as age and NIHSS cut-offs), timing of events (patients treated more quickly), timing of study (more modern technologies/standards of care may improve outcomes), and differences in outcome definitions (such as use of ECASS III (European Cooperative Acute Stroke Study) vs. the New Heidelberg Bleeding Classification to define sICH). The impact of influential outlier studies underscores the variability in the studies. While we attempted to identify and reduce this heterogeneity in the analysis, this type of heterogeneity is common in meta-analyses of stroke studies, which are mostly observational, and further underscores the need for RCTs in this area.

The findings herein suggest that device 200 and comparative device 1 may be associated with significantly lower rates of sICH and mortality compared to comparative device 2, and device 200 may be associated with significantly improved functional outcomes compared to both comparative device 1 and comparative device 2.

In each study, device 200 was prepared for delivery to the occlusion site with standard interventional techniques to access the arterial system and using angiography in order to determine the location of the occluded vessel. Once determined, a guide catheter, sheath, or balloon guide catheter was advanced as close to the occlusion as possible. A rotating hemostasis valve (RHV) was connected to the proximal end of the catheter and connected to a continuous flush system. An appropriate microcatheter was then selected and an RHV was connected to the proximal end of the microcatheter and connected to a continuous flush system. With the aid of a suitable guidewire, and using standard catheterization techniques and fluoroscopic guidance, the microcatheter was advanced up to and across the occlusion so that the distal end of the microcatheter is positioned distal of the occlusion. The guidewire was removed from the microcatheter and optionally contrast media was gently infused through the microcatheter to visualize the distal end of the occlusion. The insertion tool with the preloaded retrieval device 200 was then removed from the packaging hoop. The distal end of the insertion tool was inserted through the RHV of the microcatheter and then waited until fluid was seen exiting the proximal end of the insertion tool, confirming that device 200 was flushed. The insertion tool was then advanced until it contacted the hub of the microcatheter and the RHV was fully tightened to hold the insertion tool securely in position. The insertion tool was confirmed as being fully seated in the hub of the RHV before proceeding to advance device 200 until at least half of the shaft length of shaft 206 was inserted into the microcatheter, at which point the insertion tool was removed.

Regarding positioning and deployment, device 200 continued to be advanced towards the distal tip of the microcatheter (e.g., until the distal radiopaque tip 208 of the device 200 was aligned with the distal tip). Device 200 optionally included bands positioned on the proximal portion of shaft 206 to assist in minimizing the amount of fluoroscopic exposure required during insertion of device 200. If using a standard microcatheter (total length of 155 cm and a 7 cm RHV), then when the first band on the shaft 206 approached the RHV, while the tip of device 200 was approximately 8 cm from the distal end of the microcatheter. When the second band on the shaft 206 approached the RHV, the tip of device 200 was nearing the distal end of the microcatheter. Device 200 was then advanced in the microcatheter and positioned within the clot and left to embed for 3-5 minutes prior to withdrawal.

Device 200 was optionally supplied preloaded within an insertion tool. In such applications, the physician inserted the insertion tool into the hub of a pre-positioned microcatheter and advances the clot retrieval device forward out of the insertion tool and into the microcatheter.

During each study, device 200 tested was typically used for up to three (3) retrieval attempts. If an additional pass was made with device 200, then any captured thrombus was carefully removed therefrom, and device 200 cleaned in heparinized saline. FIG. 4 of this disclosure shows a representative overview of the study flow using the patients of each study with the clot retrieval device 200.

Patients associated with each study included those with acute ischemic stroke in anterior circulation (including distal internal carotid artery (ICA), carotid T, middle cerebral artery (MCA) segments M1 and M2) treated with endovascular treatment using the stent retriever of this disclosure as first or second line device, were retrospectively included across multiple different centers. According to the availability of the material, there were not any recommendation regarding a preferred type of stent retriever, the clot retrieval device of this disclosure was used randomly in the flow of patients.

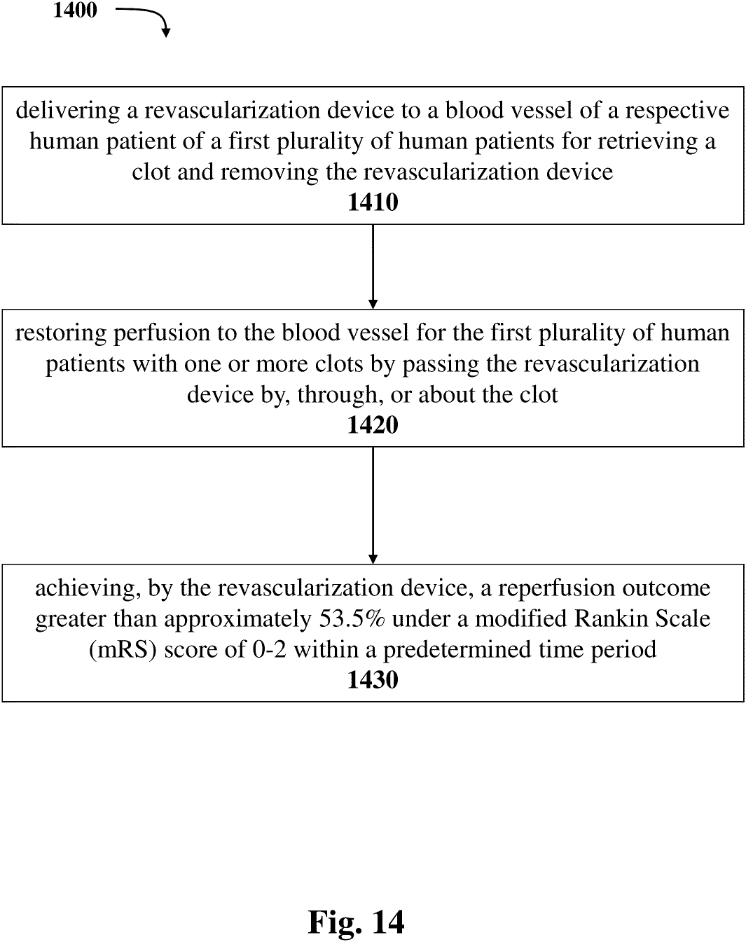

FIG. 14 depicts a method or use 1400 and can include delivering 1410 a revascularization device to a blood vessel of a respective human patient of a first plurality of human patients for retrieving a clot and removing the revascularization device; restoring 1420 perfusion to the blood vessel for the first plurality of human patients with one or more clots by passing the revascularization device by, through, or about the thrombus; and achieving 1430, by the revascularization device, a reperfusion outcome of greater than approximately 53.5%, under a modified Rankin Scale (mRS) score of 0-2 within a predetermined time period. Method or use 1400 can end after step 1430.

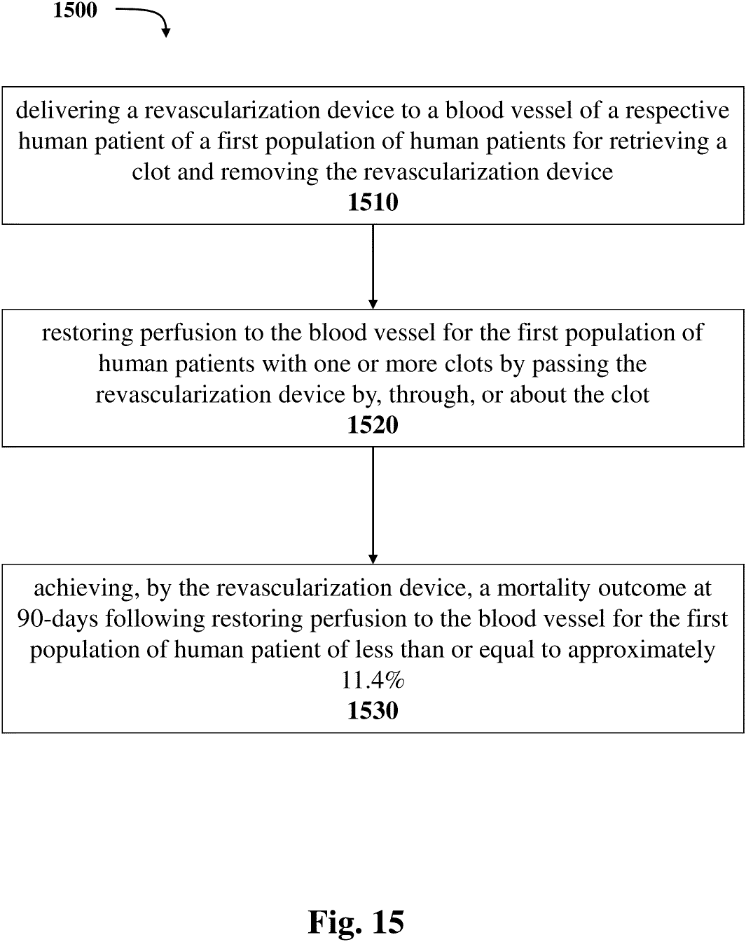

FIG. 15 depicts a method or use 1500 and can include delivering 1510 a revascularization device to a blood vessel of a respective human patient of a first plurality of human patients for retrieving a clot and removing the revascularization device; restoring 1520 perfusion to the blood vessel by passing the revascularization device by, through, or about the thrombus; and achieving 1530, by the revascularization device, a mortality outcome at 90-days following restoring perfusion to the blood vessel for the first population of human patient of less than or equal to approximately 11.4%. Method or use 1500 can end after step 1530.

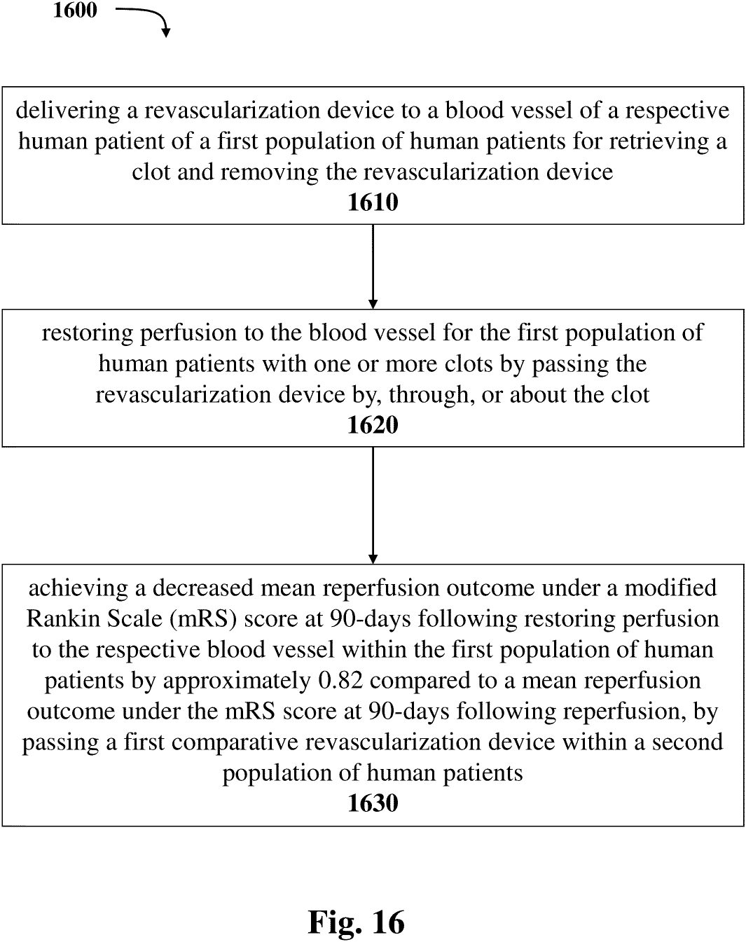

FIG. 16 depicts a method or use 1600 and can include delivering 1610 a revascularization device to a blood vessel of a respective human patient of a first plurality of human patients for retrieving a clot and removing the revascularization device; restoring 1620 perfusion to the blood vessel by passing the revascularization device by, through, or about the clot; and achieving 1630 a decreased mean reperfusion outcome under a modified Rankin Scale (mRS) score at 90-days following restoring perfusion to the respective blood vessel within the first population of human patients by approximately 0.82 compared to a mean reperfusion outcome under the mRS score at 90-days following reperfusion, by passing a first comparative revascularization device within a second population of human patients. Method or use 1600 can end after step 1630.

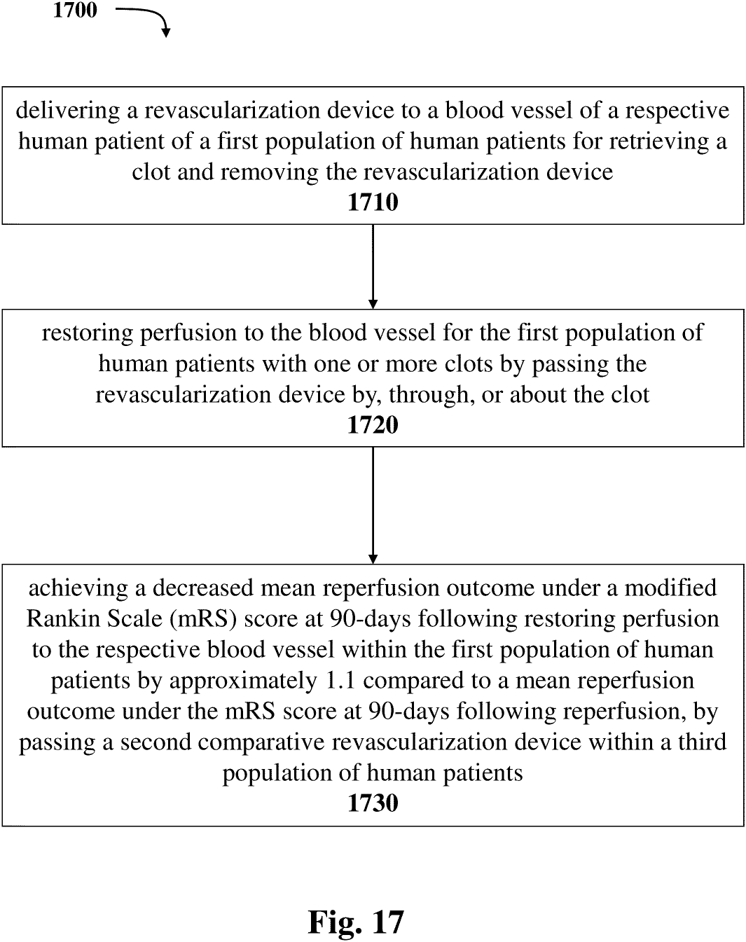

FIG. 17 depicts a method or use 1700 and can include delivering 1710 a revascularization device to a blood vessel of a respective human patient of a first plurality of human patients for retrieving a clot and removing the revascularization device; restoring 1720 perfusion to the blood vessel by passing the revascularization device by, through, or about the clot; and achieving 1730 a decreased mean reperfusion outcome under a modified Rankin Scale (mRS) score at 90-days following restoring perfusion to the respective blood vessel within the first population of human patients by approximately 1.1 compared to a mean reperfusion outcome under the mRS score at 90-days following reperfusion, by passing a second comparative revascularization device within a third population of human patients. Method or use 1700 can end after step 1730.

The device 200 and related methods of use of this disclosure demonstrated high rates of substantial reperfusion and functional independence in patients with acute ischemic stroke secondary to large-vessel occlusions. The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. It will therefore be apparent from the foregoing that while particular forms of the disclosure have been illustrated and described, various modifications can be made without departing from the spirit and scope of the disclosure and all changes that come within the meaning and range of equivalents thereof are intended to be embraced therein.

The following clauses list non-limiting embodiments of the disclosure:
1. A method of restoring blood flow in neurovasculature by removing a clot in human patients experiencing ischemic stroke, the method comprising:
   delivering a revascularization device to a blood vessel of a respective human patient of a first plurality of human patients for retrieving a clot and removing the revascularization device; and
   restoring perfusion to the blood vessel for the first plurality of human patients with one or more clots by passing the revascularization device by, through, or about the clot to achieve a reperfusion outcome greater than approximately 53.5% under a modified Rankin Scale (mRS) score of 0-2 within a predetermined time period.
2. The method of claim 1, further comprising:
   achieving, by the revascularization device, a reperfusion outcome less than or equal to approximately 64.4% under a modified Rankin Scale (mRS) score of 0-2 within a predetermined time period.
3. The method of claim 1, wherein the predetermined time period is within 90-days following restoring perfusion to the blood vessel.
4. The method of claim 1, the revascularization device comprising:
   an inner tubular body having a plurality of openings, a collapsed delivery configuration, and an expanded deployed configuration; and
   an outer tubular body at least partially overlying the inner tubular body and having a plurality of closed cell.
5. The method of claim 1, further comprising achieving a reperfusion outcome greater than approximately 19.1 % under a mRS score of 0 for the first plurality of human patients at 90-days following restoring perfusion to the blood vessel.
6. The method of claim 1, further comprising achieving a reperfusion outcome of at least approximately 21.1% under a mRS score of 0 for the first plurality of human patients at 90-days following restoring perfusion to the blood vessel.
7. The method of claim 1, further comprising achieving a reperfusion outcome greater than approximately 15.5% under a mRS score of 2 for the first plurality of human patients at 90-days following restoring perfusion to the blood vessel.
8. The method of claim 1, further comprising achieving a reperfusion outcome of at least approximately 20.6% under a mRS score of 2 for the first plurality of human patients at 90-days following restoring perfusion to the blood vessel.
9. The method of claim 1, further comprising:
   delivering a first comparative revascularization device to a blood vessel of a respective human patient of a second plurality of human patients with one or more clots;
   restoring perfusion to the respective blood vessel by passing the first comparative revascularization device by, through, or about the respective clot of the blood vessel; and
   achieving, by the revascularization device, a decreased average symptomatic intracranial hemorrhage (sICH) outcome for the first plurality of human patients by approximately 0.7% compared to the second plurality of human patients.
10. The method of claim 9, further comprising:
   achieving, by the revascularization device, an increased average reperfusion outcome under a mRS score of 0-2 at 90-days following restoring perfusion to the blood vessel for the first plurality of human patients by approximately 7.4%, by the revascularization device compared to the second plurality of human patients.
11. The method of claim 9, further comprising:
   achieving, by the revascularization device, a reduced average mortality at 90-days following restoring perfusion to the blood vessel for the first plurality of human patients by approximately 3.3% compared to the second plurality of human patients.
12. The method of claim 1, further comprising:
   delivering a second comparative revascularization device to a blood vessel of a respective human patient of a third plurality of human patients with one or more clots;
   restoring perfusion to the respective blood vessel by passing the second comparative revascularization device by, through, or about the respective clot of the blood vessel; and
   achieving, by the revascularization device, a decreased average symptomatic intracranial hemorrhage (sICH) outcome for the first plurality of human patients by approximately 3.8% compared to the third plurality of human patients.
13. The method of claim 12, further comprising
   achieving, by the revascularization device, an increased average reperfusion outcome under a mRS score of 0-2 at 90-days following restoring perfusion to the blood vessel for the first plurality of human patients by approximately 12.1%, by the revascularization device compared to the third plurality of human patients.
14. The method of claim 12, further comprising:
   achieving, by the revascularization device, a reduced average mortality at 90-days following restoring perfusion to the blood vessel for the first plurality of human patients by approximately 9.2% compared to the third plurality of human patients.
15. The method of claim 1, wherein a population size for the first plurality of human patients comprises at least 633 patients.
16. A revascularization device for restoring perfusion to a blood vessel of a respective human patient of a first population of human patients, the device comprising:
   a collapsed delivery configuration and an expanded deployed configuration;
   a framework of struts forming a porous outer body radially surrounding a porous inner body during both the collapsed delivery configuration and the expanded deployed configuration, the outer body being expandable to a radial extent to define a clot reception space;
   wherein a distal end of the inner body is located within the outer body and adjacent a distal end of the outer body and extending in the deployed configuration towards the outer body to a greater extent than the inner body; and
   wherein the revascularization device is capable of being delivered to a respective blood vessel of the first population of human patients and restoring perfusion to the blood vessel by passing the revascularization device by, though, or about a clot to achieve a reperfusion outcome greater than approximately 53.5% and less than or equal to 64.4% under a modified Rankin Scale (mRS) score of 0-2 at 90-days following restoring perfusion to the blood vessel.
17. The revascularization device of claim 16, wherein the revascularization device is capable of being delivered to a blood vessel and restoring perfusion to the blood vessel by passing the revascularization device by, though, or about the clot to achieve a mean reperfusion outcome under a modified Rankin Scale (mRS) score of at least 1.63 for the first population of human patients at 90-days following restoring perfusion to the blood vessel.
18. The revascularization device of claim 17, wherein the mean reperfusion outcome is for a first population of at least 475 human patients.
19. The revascularization device of claim 16, wherein the revascularization device is capable of being delivered to a blood vessel and restoring perfusion to the blood vessel by passing the revascularization device by, though, or about the respective clots to achieve a decreased mean reperfusion outcome under a modified Rankin Scale (mRS) score at 90-days following restoring perfusion to the respective blood vessel within a first population of human patients by approximately 0.82 compared to a mean reperfusion outcome under the mRS score at 90-days following reperfusion, by passing a first comparative revascularization device within a second population of human patients.
20. The revascularization device of claim 19, wherein the mean reperfusion outcome for the first population of human patients comprises at least 475 patients and wherein the mean reperfusion outcome for the second of human patients comprises at least 2598 patients.
21. The revascularization device of claim 16, wherein the revascularization device is capable of being delivered to a blood vessel and restoring perfusion to the blood vessel by passing the revascularization device by, though, or about the respective clot to achieve a decreased mean reperfusion outcome under a modified Rankin Scale (mRS) score at 90-days following restoring perfusion to the respective blood vessel within a first population of human patients by approximately 1.1 compared to a mean reperfusion outcome under the mRS score at 90-days following reperfusion, by passing a second comparative revascularization device within a third population of human patients.
22. The revascularization device of claim 21, wherein the mean reperfusion outcome for the first population of human patients comprises at least 475 patients and wherein the mean reperfusion outcome for the third population of human patients comprises at least 2893 patients.
23. A method of restoring blood flow in neurovasculature by removing a clot in human patients experiencing ischemic stroke, the method comprising:
   delivering a revascularization device to a blood vessel of a respective human patient of a first population of human patients for retrieving a clot and removing the revascularization device; and
   restoring perfusion to the blood vessel for the first population of human patients with one or more clots by passing the revascularization device by, through, or about the clot to achieve a mortality outcome at 90-days following restoring perfusion to the blood vessel for the first population of human patient of less than or equal to approximately 11.4%.
24. The method of claim 23, further comprising achieving a mean symptomatic intracranial hemorrhage (sICH) outcome of approximately 3.9% for the first population of human patients.
25. The method of claim 23, the revascularization device comprising:
   an inner tubular body having a plurality of openings, a collapsed delivery configuration, and an expanded deployed configuration; and
   an outer tubular body at least partially overlying the inner tubular body and having a plurality of closed cell.
26. The method of claim 23, further comprising achieving a mean reperfusion outcome under a modified Rankin Scale (mRS) score of at least 1.63 for the first population of human patients at 90-days following restoring perfusion to the blood vessel.
27. The method of claim 23, further comprising achieving a decreased mean reperfusion outcome under a modified Rankin Scale (mRS) score at 90-days following restoring perfusion to the respective blood vessel within the first population of human patients by approximately 0.82 compared to a mean reperfusion outcome under the mRS score at 90-days following reperfusion, by passing a first comparative revascularization device within a second population of human patients.
28. The method of claim 23, further comprising achieving a decreased mean reperfusion outcome under a modified Rankin Scale (mRS) score at 90-days following restoring perfusion to the respective blood vessel within the first population of human patients by approximately 1.1 compared to a mean reperfusion outcome under the mRS score at 90-days following reperfusion, by passing a second comparative revascularization device within a third population of human patients.

What is claimed is:

1. A method of restoring blood flow in neurovasculature by removing a clot in human patients experiencing ischemic stroke, the method comprising:
delivering a revascularization device to a blood vessel of a respective human patient of a first plurality of human patients for retrieving a clot and removing the revascularization device; and
restoring perfusion to the blood vessel for the first plurality of human patients with one or more clots by passing the revascularization device by, through, or about the clot to achieve a reperfusion outcome greater than approximately 53.5% under a modified Rankin Scale (mRS) score of 0-2 within a predetermined time period.

2. The method of claim 1, further comprising:
achieving, by the revascularization device, a reperfusion outcome less than or equal to approximately 64.4% under a modified Rankin Scale (mRS) score of 0-2 within a predetermined time period.

3. The method of claim 1, wherein the predetermined time period is within 90-days following restoring perfusion to the blood vessel.

4. The method of claim 1, the revascularization device comprising:
an inner tubular body having a plurality of openings, a collapsed delivery configuration, and an expanded deployed configuration; and
an outer tubular body at least partially overlying the inner tubular body and having a plurality of closed cell.

5. The method of claim 1, further comprising achieving a reperfusion outcome greater than approximately 19.1% under a mRS score of 0 for the first plurality of human patients at 90-days following restoring perfusion to the blood vessel.

6. The method of claim 1, further comprising achieving a reperfusion outcome of at least approximately 21.1% under a mRS score of 0 for the first plurality of human patients at 90-days following restoring perfusion to the blood vessel.

7. The method of claim 1, further comprising achieving a reperfusion outcome greater than approximately 15.5% under a mRS score of 2 for the first plurality of human patients at 90-days following restoring perfusion to the blood vessel.

8. The method of claim 1, further comprising achieving a reperfusion outcome of at least approximately 20.6% under a mRS score of 2 for the first plurality of human patients at 90-days following restoring perfusion to the blood vessel.

9. The method of claim 1, further comprising:
delivering a first comparative revascularization device to a blood vessel of a respective human patient of a second plurality of human patients with one or more clots;
restoring perfusion to the respective blood vessel by passing the first comparative revascularization device by, through, or about the respective clot of the blood vessel; and
achieving, by the revascularization device, a decreased average symptomatic intracranial hemorrhage (sICH) outcome for the first plurality of human patients by approximately 0.7% compared to the second plurality of human patients.

10. The method of claim 9, further comprising:
achieving, by the revascularization device, an increased average reperfusion outcome under a mRS score of 0-2 at 90-days following restoring perfusion to the blood vessel for the first plurality of human patients by approximately 7.4%, by the revascularization device compared to the second plurality of human patients.

11. The method of claim 9, further comprising:
achieving, by the revascularization device, a reduced average mortality at 90-days following restoring perfusion to the blood vessel for the first plurality of human patients by approximately 3.3% compared to the second plurality of human patients.

12. The method of claim 1, further comprising:
delivering a second comparative revascularization device to a blood vessel of a respective human patient of a third plurality of human patients with one or more clots;
restoring perfusion to the respective blood vessel by passing the second comparative revascularization device by, through, or about the respective clot of the blood vessel; and
achieving, by the revascularization device, a decreased average symptomatic intracranial hemorrhage (sICH) outcome for the first plurality of human patients by approximately 3.8% compared to the third plurality of human patients.

13. The method of claim 12, further comprising
achieving, by the revascularization device, an increased average reperfusion outcome under a mRS score of 0-2 at 90-days following restoring perfusion to the blood vessel for the first plurality of human patients by approximately 12.1%, by the revascularization device compared to the third plurality of human patients.

14. The method of claim 12, further comprising:
achieving, by the revascularization device, a reduced average mortality at 90-days following restoring perfusion to the blood vessel for the first plurality of human patients by approximately 9.2% compared to the third plurality of human patients.

15. The method of claim 1, wherein a population size for the first plurality of human patients comprises at least 633 patients.

16. A method of restoring blood flow in neurovasculature by removing a clot in human patients experiencing ischemic stroke, the method comprising:
delivering a revascularization device to a blood vessel of a respective human patient of a first population of human patients for retrieving a clot and removing the revascularization device; and
restoring perfusion to the blood vessel for the first population of human patients with one or more clots by passing the revascularization device by, through, or about the clot to achieve a mortality outcome at 90-days following restoring perfusion to the blood vessel for the first population of human patient of less than or equal to approximately 11.4%.

17. The method of claim 16, further comprising achieving a mean symptomatic intracranial hemorrhage (sICH) outcome of approximately 3.9% for the first population of human patients.

18. The method of claim 16, further comprising achieving a mean reperfusion outcome under a modified Rankin Scale (mRS) score of at least 1.63 for the first population of human patients at 90-days following restoring perfusion to the blood vessel.

19. The method of claim 16, further comprising achieving a decreased mean reperfusion outcome under a modified Rankin Scale (mRS) score at 90-days following restoring perfusion to the respective blood vessel within the first population of human patients by approximately 0.82 compared to a mean reperfusion outcome under the mRS score at 90-days following reperfusion, by passing a first comparative revascularization device within a second population of human patients.

20. The method of claim 16, further comprising achieving a decreased mean reperfusion outcome under a modified Rankin Scale (mRS) score at 90-days following restoring perfusion to the respective blood vessel within the first population of human patients by approximately 1.1 compared to a mean reperfusion outcome under the mRS score at 90-days following reperfusion, by passing a second comparative revascularization device within a third population of human patients.
